SEQUENCE LISTING (4 sequences)

SEQ ID NO: 1 - DNA, 18 bp, Artificial Sequence
  PCR primer
agcagagctcgtttagtg

SEQ ID NO: 2 - DNA, 18 bp, Artificial Sequence
  PCR primer
ggaggcggcgacattgtc

SEQ ID NO: 3 - DNA, 25 bp, Artificial Sequence
  PCR primer
cacgatacacctgactagctgggtg

SEQ ID NO: 4 - DNA, 25 bp, Artificial Sequence
  PCR primer
catcacccacaggctagcgccaact

(19) United States
(12) Patent Application Publication
(10) Pub. No.: US 2016/0175308 A1
(43) Pub. Date: Jun. 23, 2016

(54) THERAPEUTIC AGENTS FOR MODULATING THYMIC FUNCTION AND/OR GROWTH AND/OR TREATING VARIOUS DISORDERS

(71) Applicant: UCL BUSINESS PLC, London (GB)

(72) Inventor: Adam Giangreco, London (GB)

(73) Assignee: UCL BUSINESS PLC, London (GB)

(21) Appl. No.: 14/786,653

(22) PCT Filed: May 2, 2014
(86) PCT No.: PCT/GB2014/051381

§ 371 Date: Oct. 23, 2015

(30) Foreign Application Priority Data
May 2, 2013 (GB) 1307967.8

(51) Int. Cl. A61K 31/517; A61K 31/439; A61K 31/436

(52) U.S. Cl. CPC A61K 31/517; A61K 31/436; A61K 31/439

(57) ABSTRACT

The present disclosure relates to a therapeutic agent for use in a method for modulating the function and/or growth of a thymus in a subject, wherein the therapeutic agent comprises an HER2 or HER1 pathway antagonist or agonist, and/or a CCR/CCL5 antagonist the method involving administering the therapeutic agent to the subject. Also disclosed herein is a therapeutic agent for use in a method for treating a disorder in a subject, the disorder selected from systemic autoimmunity, peripheral autoimmunity and Systemic Lupus Erythematosus,

### BACKGROUND

The ability of the immune system to mount an effective response to injury, infection and disease depends on maintaining an abundant, naïve T cell population within the thymus. Unfortunately, thymic atrophy and involution (the shrinking of the thymus) occurs naturally throughout life and results in decreased output of naïve T cells from middle age. Thymic atrophy and involution are also greatly accelerated in malnourished individuals, post chemo- and radiotherapy, and following infection with HIV (Haynes and Hale, 1998). It is now well accepted that thymic atrophy and involution determine susceptibility to infection, influence cancer resistance, can cause systemic and peripheral inflammation resulting in autoimmunity and are major contributing factors to human morbidity and mortality (Heng, et al 2010). Separately, 75% of cases of the rare autoimmune disorder myasthenia gravis (MG) are caused by excessive thymic growth or formation of a benign thymus tumour (thymoma). Thus, there is a clear clinical need for therapies to modulate thymic growth, atrophy and involution.

Current therapies for modulating thymic atrophy and involution are generally not clinically or commercially viable. These include administration of recombinant human proteins such as keratinocyte growth factor (KGF) and interleukin 7 (IL-7) or chemical or physical castration of aging individuals (Aspinall and Mitchell, 2008). Drawbacks to recombinant protein administration include a very high cost to manufacture, a requirement for continuous or repeated intravenous administration due to the short half-life of KGF and IL-7 in vivo, and significant reported side effects including bone loss and increased autoimmunity. The drawbacks to castration are additionally significant and include sterility, impotence and loss of sex drive. Current treatment for MG is by immunosuppressive drugs or surgical thymectomy, each of which also typically require ongoing treatment throughout the patient's life.

### SUMMARY OF THE INVENTION

In an aspect, the present invention provides a therapeutic agent for use in a method for modulating the function and/or growth of a thymus in a subject, wherein the therapeutic agent comprises an HER2 or HER1 pathway antagonist or agonist the method involving administering the therapeutic agent to the subject. In an aspect, the present invention provides a therapeutic agent for use in a method for modulating the function and/or growth of a thymus in a subject, wherein the therapeutic agent a CCR/CCL5 antagonist, the method involving administering the therapeutic agent to the subject. In an embodiment, the method involves treating thymic atrophy and/or involution in the subject, and wherein the agent may comprise an HER2 or HER1 pathway antagonist. In an embodiment, the method involves treating a hyperactive thymus and/or excessive thymic growth in the subject, and the agent is a HER2 or HER1 pathway agonist and/or a CCR/CCL5 antagonist. In an embodiment, the method involves treating systemic or peripheral autoimmunity using an HER2 or HER1 pathway antagonist or agonist.

In an aspect, the present invention provides a therapeutic agent for use in a method for treating a disorder in a subject, the disorder selected from systemic autoimmunity, peripheral autoimmunity and Systemic Lupus Erythematosus, wherein the therapeutic agent is as described herein, for example is or comprises an HER2 or HER1 pathway antagonist or agonist the method involving administering the therapeutic agent to the subject. The HER2 or HER1 pathway antagonist or agonist may be as defined herein. Peripheral autoimmunity may be defined as ‘inflammation and auto reactive leukocyte infiltration to peripheral organs such as the skin’. Systemic autoimmunity may be defined as ‘inflammation and auto reactive leukocyte infiltration to internal organs and tissues’. The systemic autoimmunity may be selected from systemic lupus erythematosus, IPEX or XLAAD syndrome, myasthenia gravis, and APECED syndrome The peripheral autoimmunity may be selected from systemic lupus erythematosus, psoriasis, and autoimmune alopecia. The subject having the disorder selected from systemic autoimmunity, peripheral autoimmunity and Systemic Lupus Erythematosus may or may not be suffering from any of thymic atrophy, thymic involution, a hyperactive thymus and excessive thymic growth. XLAAD can indicate X-linked autoimmunity and allergic dysregulation, sometimes termed IPEX (immune dysfunction, polyendocrinopathy, and enteropathy, X-linked)). APECED can indicate Autoimmune polyendocrine syndrome type 1.

In an aspect, the present invention provides a therapeutic agent for use in a method for treating a disorder in a subject, the disorder selected from systemic autoimmunity, peripheral autoimmunity and Systemic Lupus Erythematosus, wherein the therapeutic agent is selected from lapatinib, gefitinib, erlotinib, neratinib, afatinib, CP724714, CP654577, canertinib, BIBW2992, AG1478, rapamycin, perifosine, pelitinib, Arry334543, CL-387785, AV-412,AEE788, CGP-59326A, PKI-166, HKI-357, BMS-599626, PX866, SDZ-RAD, ARRY142886, Selumetinib, Sorafenib, BIBW2948, HKI272, Ruxolitinib and any salt, base or prodrug form thereof. In an aspect, the present invention provides a therapeutic agent for use in a method for treating a disorder in a subject, the disorder selected from systemic autoimmunity, peripheral autoimmunity and Systemic Lupus Erythematosus, wherein the therapeutic agent is or comprises an antibody selected from cetuximab, trastuzumab, pertuzumab, peruzumab, and panitumumab, and any salt, base or prodrug form thereof. The systemic autoimmunity may be selected from systemic lupus erythematosus, IPEX or XLAAD syndrome, myasthenia gravis, and APECED syndrome The peripheral autoimmunity may be selected from systemic lupus erythematosus, psoriasis, and autoimmune alopecia.

In an aspect, the present invention provides a therapeutic agent for use in a method for treating a disorder in a subject, the disorder selected from systemic autoimmunity, peripheral autoimmunity and Systemic Lupus Erythematosus, wherein the therapeutic agent is selected from lapatinib, gefitinib, erlotinib, neratinib and afatinib, and any salt, base or prodrug form thereof. The systemic autoimmunity may be selected from systemic lupus erythematosus, IPEX or XLAAD syndrome, myasthenia gravis, and APECED syndrome The peripheral autoimmunity may be selected from systemic lupus erythematosus, psoriasis, and autoimmune alopecia.

In an aspect, the present invention provides a therapeutic agent for use in a method for treating a disorder in a subject, the disorder selected from systemic autoimmunity, peripheral autoimmunity and Systemic Lupus Erythematosus, wherein the therapeutic agent is selected from rapamycin and perifosine and any salt, base or prodrug form thereof. The systemic autoimmunity may be selected from systemic lupus erythematosus, IPEX or XLAAD syndrome, myasthenia gravis, and APECED syndrome The peripheral autoimmunity may be selected from systemic lupus erythematosus, psoriasis, or autoimmune alopecia

In an aspect, the present invention provides a method for modulating the function and/or growth of a thymus in a subject, the method involving administering a therapeutic agent comprising an HER2 or HER1 pathway antagonist or agonist to the subject.

In an aspect, the present invention provides a therapeutic agent for use in a method for modulating the function and/or growth of a thymus in a subject, wherein the therapeutic agent is or comprises a compound according to formula (I) and/or an antibody that is an HER1 or HER2 pathway antagonist:

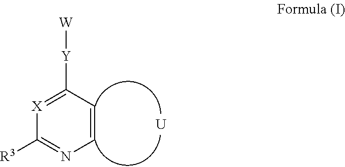

X is N, CH or C—C≡N;
Y is a group CH₂, Z(CH₂), (CH₂)Z, or Z, in which Z is O, S(O)ₘ wherein m is 0, 1 or 2, or NR^{a} wherein R^{a} is hydrogen or a C₁₋₈ alkyl group;
W is an optionally substituted aromatic monocyclic or aromatic bicyclic ring;

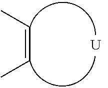

is an optionally substituted, fused 5, 6 or 7-membered aromatic ring, optionally containing 1 to 5 heteroatoms which may be the same or different and which are selected from N, O or S(O)_{m′} wherein m′ is 0, 1 or 2, the heterocyclic ring containing a total of 1, 2 or 3 double bonds inclusive of the bond in the pyridine or pyrimidine ring;
R³ is selected from hydrogen, halo, trifluoromethyl, C₁₋₄ alkyl and C₁₋₄ alkoxy;

and any salt, base or prodrug form thereof. The compound according to formula (I) may be an HER1 or HER2 pathway antagonist, and used in the method for treating thymic atrophy and/or involution in the subject. In some embodiments, the therapeutic agent is selected from lapatinib, gefitinib, erlotinib, neratinib, afatinib, CP724714, CP654577, canertinib, BIBW2992, AG1478, rapamycin, perifosine, pelitinib, Arry334543, CL-387785, AV-412,AEE788, CGP-59326A, PKI-166, HKI-357, BMS-599626, PX866, SDZ-RAD, ARRY142886, Selumetinib, Sorafenib, BIBW2948, HKI272, Ruxolitinib and any salt, base or prodrug form thereof, and optionally the therapeutic agent is used in the method for treating thymic atrophy and/or involution in the subject. Optionally, the therapeutic agent is or comprises an antibody selected from cetuximab, trastuzumab, pertuzumab, peruzumab, and panitumumab and the therapeutic agent is for treating thymic atrophy and/or involution in the subject.

The compound according to formula (I) may be an HER1 or HER2 pathway antagonist, and used in the method for treating thymic atrophy and/or involution in the subject. In some embodiments, the therapeutic agent is selected from lapatinib, gefitinib, erlotinib, neratinib, afatinib, CP724714, CP654577, canertinib, BIBW2992, AG1478, rapamycin, perifosine, pelitinib, Arry334543, CL-387785, AV-412,AEE788, CGP-59326A, PKI-166, HKI-357, BMS-599626, PX866, SDZ-RAD, ARRY142886, Selumetinib, Sorafenib, BIBW2948, HKI272, Ruxolitinib and any salt, base or prodrug form thereof, and optionally the therapeutic agent is used in the method for treating thymic atrophy and/or involution in the subject. Optionally, the therapeutic agent is or comprises an antibody selected from cetuximab, trastuzumab, pertuzumab, peruzumab, and panitumumab and the therapeutic agent is for treating thymic atrophy and/or involution in the subject.

The compound according to formula (I) may be an HER1 or HER2 pathway antagonist, and used in the method for treating thymic atrophy and/or involution in the subject. In some embodiments, the therapeutic agent is selected from lapatinib, gefitinib, erlotinib, neratinib and afatinib, and any salt, base or prodrug form thereof, and optionally the therapeutic agent is used in the method for treating thymic atrophy and/or involution in the subject. Optionally, the therapeutic agent is or comprises an antibody selected from cetuximab, trastuzumab and peruzumab, and the therapeutic agent is for treating thymic atrophy and/or involution in the subject. In some embodiments, the therapeutic agent is selected from CP724714, CP654577, canertinib, BIBW2992, AG1478, rapamycin, perifosine, pelitinib, Arry334543, CL-387785, AV-412,AEE788, CGP-59326A, PKI-166, HKI-357, BMS-599626, PX866, SDZ-RAD, ARRY142886, Selumetinib, Sorafenib, BIBW2948, HKI272, Ruxolitinib rapamycin, perifosine, and any salt, base or prodrug form thereof, and optionally the therapeutic agent is used in the method for treating thymic atrophy and/or involution in the subject.

In some embodiments, the therapeutic agent is selected from rapamycin and perifosine, and any salt, base or prodrug form thereof, and optionally the therapeutic agent is used in the method for treating thymic atrophy and/or involution in the subject.

Optionally, the therapeutic agent is or comprises an antibody selected from pertuzumab and panitumumab and the therapeutic agent is for treating thymic atrophy and/or involution in the subject.

In an aspect, the present invention provides a composition comprising the therapeutic agent as described herein. The composition may comprise the therapeutic agent and a pharmaceutical acceptable carrier or excipient, and the composition may be for use in a method for modulating the function and/or growth of a thymus in a subject, wherein the therapeutic agent comprises an HER2 or HER1 pathway antagonist or agonist and/or a CCR/CCL5 antagonist, the method involving administering the therapeutic agent to the subject. The composition may be for treating thymic atrophy and/or involution in a subject, and the therapeutic agent may be or comprise an HER2 or HER1 pathway antagonist. The composition may be for treating a hyperactive thymus and/or excessive thymic growth in a subject, and the therapeutic agent may be a HER2 or HER1 pathway agonist and/or a CCR/CCL5 antagonist. The CCR/CCL5 antagonist may comprise or be maraviroc.

In an aspect, the present invention provides a method for modulating the function and/or growth of a thymus in a subject, the method involving administering a therapeutic agent to the subject, wherein the therapeutic agent comprises an HER2 or HER1 pathway antagonist or agonist. In an embodiment, the method is for treating thymic atrophy and/or involution in the subject, and wherein the agent comprises an HER2 or HER1 pathway antagonist. In an embodiment, the method is for treating a hyperactive thymus and/or excessive thymic growth in a subject, and the therapeutic agent is or comprises a HER2 or HER1 pathway agonist and/or a CCR/CCL5 antagonist.

The present inventors have devised methods for modulating thymic growth, atrophy and involution by regulating HER2 or HER1 pathway activation. The present inventors found that transgene-mediated activation of the HER2 pathway specifically within thymic epithelial cells causes thymic atrophy as well as significant inflammation indicative of autoimmunity of the peripheral(dermatological), respiratory, and musculoskeletal systems. These characteristics are consistent with the transgene mediated activation of HER2 representing a model of human autoimmune disease, notably IPEX/XLAAD, systemic lupus erythematosus (SLE), and APECED syndromes. As illustrated in the Examples below, HER2 activation caused very rapid (within 4 days) and severe (>75% loss of mass) thymic atrophy in young adult mice (6-12 weeks of age; FIG. 1). In the Examples, it is shown that HER2 activation was characterised by complete loss of cortical thymic structures as well as near-complete depletion of naïve T cells (CD4/8 double positive (DP-T) cells). The present inventors found that switching off transgene-mediated HER2 activation after 4 days resulted in complete restoration of thymic mass and reversal of atrophy/involution and normalization of systemic and peripheral inflammation consistent with reduced autoimmunity after 28 days. This was accompanied both by restoration of cortical thymic structures as well as normal naïve T cell abundance. These data strongly implicate the HER2 pathway as a regulator of thymic growth, atrophy and involution as well as peripheral and systemic inflammation and autoimmunity consistent with human autoimmune diseases including IPEX/XLAAD, SLE, and APECED syndromes.

The present inventors also found that administering a HER2 and HER1 pathway antagonist can increase thymus weight. This is illustrated in the Examples, for example with the HER2 and HER1 pathway antagonist Lapatinib (having the trade name Tykerb/Tyverb (TM)).

### BRIEF DESCRIPTION OF THE FIGURES

FIG. 1 is a graph that illustrates that cortical and medullary thymus epithelial cells express Her1 and Her2.

FIG. 2 illustrates that thymic epithelial Her2 activation caused rapid onset, reversible thymus involution

FIG. 3 illustrates that Her2 activation causes depletion of thymic cortex and immature (DP-T) T lymphocytes.

FIG. 4 illustrates that thymic epithelial Her2 activation alters T lymphocyte development.

FIG. 5 illustrates that thymic epithelial Her2 activation causes depletion of T lymphocytes from peripheral blood.

FIG. 6 illustrates that thymic epithelial Her2 activation alters thymic stromal cell cytokine abundance.

FIG. 7 illustrates that treatment with lapatinib increases thymus size in elderly animals.

FIG. 8 illustrates that lapatinib exposure reverses thymus involution in elderly animals.

FIG. 9 illustrates that lapatinib increases thymus immature T lymphocyte production in elderly individuals.

FIG. 10 illustrates that lapatinib exposure affects thymus T lymphocyte development.

FIG. 11 illustrates a model depicting pharmacological modulators of thymus function in elderly and immunocompromised individuals and their potential mechanisms of action.

FIGS. 12A to 12D illustrate that experimentally induced HER2 activation causes reversible depletion of regulatory T cell (Treg, CD4SP, CD3+CD25+) populations from the thymus.

FIGS. 13A to 13D illustrate that thymic epithelial Her2 activation causes an increase in peripheral lymph node cellularity but does not alter lymph node T cell phenotypes.

FIGS. 14A and 14B illustrate that thymic epithelial Her2 activation and subsequent thymus atrophy causes reduced CCL5 and CXCR9 gene expression in non-hematopoietic thymus cells.

FIGS. 15A and 15B illustrates that treatment with rapamycin prevents experimentally induced, epithelial Her2-dependent thymus atrophy.

FIGS. 16A to 16D illustrate that rapamycin treatment prevents Her2 activation-dependent changes to immature thymocyte abundance.

FIGS. 17A to 17D illustrates a mechanistic model depicting how Her1/2 activation inhibits normal Treg, and subsequently CD4 and CD8 T cell development within the thymus.

### DETAILED DESCRIPTION

The present invention provides the aspects and embodiments described above. Optional and preferred features are described below. These optional and preferred features are applicable to all aspects and embodiments unless otherwise stated. Any optional or preferred feature may be combined with any other optional or preferred feature unless otherwise stated.

In an aspect, there is provided a therapeutic agent for use in a method for modulating the function and/or growth of a thymus in a subject, wherein the therapeutic agent comprises an HER2 or HER1 pathway antagonist or agonist the method involving administering the therapeutic agent to the subject.

In an aspect, there is also provided a method for modulating the function and/or growth of a thymus in a subject, the method involving administering a therapeutic agent comprising an HER2 or HER1 pathway antagonist or agonist to the subject.

The method may involve administering the agent for treating thymic atrophy and/or involution in the subject, and wherein the agent comprises an HER2 or HER1 pathway antagonist.

In an embodiment, the agent is a HER2 or HER1 pathway agonist and/or a CCR/CCL5 antagonist for treating a hyperactive thymus and/or excessive thymic growth in a subject, and optionally the agent is maraviroc. Optionally, the subject is also suffering from a disorder selected from thymoma, myasthenia Gravis, and thymic carcinoma.

Optionally, the agent comprises a compound according to formula (I) and/or an antibody that is an HER1 or HER2 pathway antagonist:

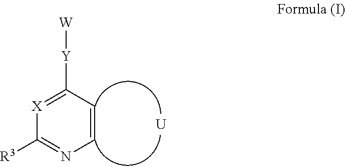

X is N, CH or C—C≡N;
Y is a group selected from NR^{a} wherein R^{a} is hydrogen or a C₁₋₈ alkyl group; CH₂, Z(CH₂), (CH₂)Z, and Z, in which Z is O, S(O)ₘ wherein m is 0, 1 or 2;
W is an optionally substituted aromatic monocyclic or aromatic bicyclic ring;
wherein W is optionally substituted by R⁴ and/or R⁵;

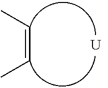

is an optionally substituted fused 5, 6 or 7-membered aromatic ring, optionally containing 1 to 5 heteroatoms which may be the same or different and which are selected from N, O or S(O)_{m′} wherein m′ is 0, 1 or 2, the heterocyclic ring containing a total of 1, 2 or 3 double bonds inclusive of the bond in the pyridine or pyrimidine ring;
wherein

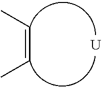

is optionally substituted by R¹ and/or R²;
R¹ and R² are the same or different and independently selected from halo, hydroxyl, optionally substituted C₁₋₈ alkyl, optionally substituted C₂₋₈ alkenyl, optionally substituted C₂₋₈ alkynyl, optionally substituted C₁₋₈ alkoxy, di-C₁₋₈ alkoxy, carboxy, carbonyl, C₁₋₈ alkylcarbonyl, C₁₋₈ alkoxycarbonyl, carbamoyl, alkylcarbamoyl, dialkylcarbamoyl, carbamyl, trifluoromethyl, ether, nitro, cyano, amino, hydroxyamino, aminocarbonyl, alkylamino, dialkylamino, di-[(C₁₋4)alkyl]amino-(C₂₋₄)alkoxy, alkylaminocarbonyl, optionally substituted furyl (e.g. [(C₁₋₄)alkylsulfonyl(C₁₋₄)alkylamino)alkyl-furyl]), optionally substituted phenyl, optionally substituted phenoxy, phenyl-V-alkyl, wherein V is selected from a single bond, O, S and NH, optionally substituted phenyl-(C₁₋₄)alkoxy, optionally substituted guanidine, optionally substituted ureido, optionally substituted pyridinyl, optionally substituted pyrimidinyl, optionally substituted pyrrolidinyl, pyrrolidin-1-yl-(C₂₋₄)alkoxy, optionally substituted piperidino, piperidino-(C₂₋₄)alkoxy, optionally substituted morpholino, morpholino-(C₁₋₄)alkoxy, optionally substituted piperazinyl, piperazin-1-yl(C₂₋₄)alkoxy, 4-(C₁₋₄)alkylpiperazin-1-yl-(C₂₋₄)alkoxy, optionally substituted imidazolyl, imidazol-1-yl(C₂₋₄)alkoxy, di-[(C₁₋₄)alkoxy-(C₂₋₄)alkyl]amino-(C₂₋₄)alkoxy, thiamorpholino-(C₂₋₄)alkoxy, 1-oxothiamorpholino-(C₂₋₄)alkoxy or 1,1-dioxothiamorpholino-(C₂₋₄)alkoxy, alkylthio, alkylsulphinyl, alkylsulphonyl, (E)-dimethylamino(but-2-enamide), optionally substituted (tetrahydro-furan-3-yl)-oxy;
R³ is selected from hydrogen, halo, trifluoromethyl, C₁₋₄ alkyl and C₁₋₄ alkoxy;
R⁴ and R⁵ are the same or different and independently selected from hydrogen, halo, hydroxyl, optionally substituted C₁₋₈ alkyl, optionally substituted C₂₋₈ alkenyl, optionally substituted C₂₋₈ alkynyl, optionally substituted C₁₋₈ alkoxy, di-C₁₋₈ alkoxy, carboxy, carbonyl, C₁₋₈ alkylcarbonyl, C₁₋₈ alkoxycarbonyl, carbamoyl, alkylcarbamoyl, dialkylcarbamoyl, carbamyl, trifluoromethyl, ether, nitro, cyano, amino, hydroxyamino, aminocarbonyl, alkylamino, dialkylamino, di-[(C₁₋4)alkyl]amino-(C₂₋₄)alkoxy, alkylaminocarbonyl, optionally substituted furyl (e.g. [(C₁₋₄)alkylsulfonyl(C₁₋₄)alkylamino)alkyl-furyl]), optionally substituted phenyl, optionally substituted phenyl(C₁₋₈)alkoxy, optionally substituted phenoxy, phenyl-V-alkyl, wherein V is selected from a single bond, O, S and NH, optionally substituted phenyl-(C₁₋₄)alkoxy, optionally substituted guanidine, optionally substituted ureido, optionally substituted pyridinyl, optionally substituted pyrimidinyl, optionally substituted pyrrolidinyl, pyrrolidin-1-yl-(C₂₋₄)alkoxy, optionally substituted piperidino, piperidino-(C₂₋₄)alkoxy, optionally substituted morpholino, morpholino-(C₁₋₄)alkoxy, optionally substituted piperazinyl, piperazin-1-yl(C₂₋₄)alkoxy, 4-(C₁₋₄)alkylpiperazin-1-yl-(C₂₋₄)alkoxy, optionally substituted imidazolyl, imidazol-1-yl(C₂₋₄)alkoxy, di-[(C₁₋₄)alkoxy-(C₂₋₄)alkyl]amino-(C₂₋₄)alkoxy, thiamorpholino-(C₂₋₄)alkoxy, 1-oxothiamorpholino-(C₂₋₄)alkoxy or 1,1-dioxothiamorpholino-(C₂₋₄)alkoxy, alkylthio, alkylsulphinyl, alkylsulphonyl;
and any salt, base or prodrug form thereof.

Optionally, W is selected from any of the following optionally substituted groups: phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3-dihydro-1H-benzimidazolyl or 1H-benzotriazolyl group.

Optionally, the compound is of formula (II);

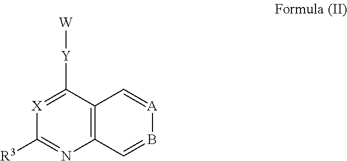

wherein W, X, Y, Z, m, R¹, R², R³, R⁴ and R⁵ are as defined above in respect of formula (I);
A and B are each independently selected from C—R₁, C—R₂ and CH, and optionally at least one of A and B is C—R₁, C—R₂, and optionally A is C—R₁ and B is C—R₂.
and any salt, base or prodrug form thereof.

Optionally, the compound is of formula (III);

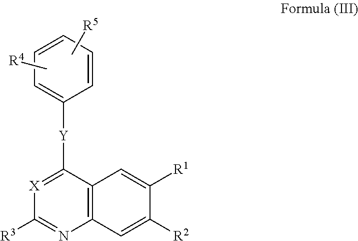

X, Y, Z, m, R¹, R², R³, R⁴ and R⁵ are as defined above in respect of formula (I) or formula (II);

Optionally, in any of the formulae, X is N, CH or C—C≡N.

Optionally, in any of the formulae, Y is NH.

Optionally, in any of the formulae, R³ is hydrogen.

Optionally, in any of the formulae, R¹ is 5-[(2-methylsulfonylethylamino)methyl]-2-furyl.

Optionally, in any of the formulae, R¹ is methoxy and R² is (3-morpholin-4ylpropoxy).

Optionally, halogen or halo may be selected from fluorine, chlorine, bromine and iodine.

Optionally, unless otherwise stated, each “alk”, “alkyl” or similar terms in the formulae herein may each independently be selected from a C₁₋₈ alkyl, optionally C₁₋₅ alkyl, optionally C₁₋₄ alkyl, optionally methyl, ethyl, propyl and butyl, and may be a branched, straight chain, and optionally substituted.

A group may be described as optionally substituted herein. Such a group may have one or more substituents thereon. The one or more substituents may, for example, be selected from halogen, nitro, cyano, hydroxy, optionally substituted alkoxy, optionally substituted amino, carboxy, alkoxycarbonyl, methylenedioxy, ethylenedioxy, optionally substituted alkylcarbonyloxy and optionally substituted arylalkoxy, alkyl e.g. C₁₋₈ alkyl, alkenyl, e.g. C₂₋₈ alkenyl, and alkynyl, e.g. C₂₋₈ alkynyl, and other substituents described herein.

Formula (I)

The following relate to possible embodiments of formula (I).

Optionally, in formula (I), “U” is a fused, optionally substituted benzene ring. In some embodiments, W is an optionally substituted benzene ring. In other embodiments, X is N, CH or C—C≡N. In another embodiment, Y is NH.

In one embodiment, X is N, CH or C—C≡N and “U” is a fused, optionally substituted benzene ring. In another embodiment, X is N, CH or C—C≡N and R³ is hydrogen.

In a further embodiment, X is N, CH or C—C≡N, “U” is a fused, optionally substituted benzene ring and R³ is hydrogen. In a further embodiment, “U” is a fused, optionally substituted benzene ring, W is an optionally substituted benzene ring and X is N, CH or C—C≡N. In another embodiment, “U” is a fused, optionally substituted benzene ring, X is N, CH or C—C≡N and Y is NH. In another embodiment, X is N, CH or C—C≡N, Y is NH and W is an optionally substituted benzene ring.

In a further embodiment, X is N, CH or C—C≡N, Y is NH, “U” is a fused optionally substituted benzene ring, and W is an optionally substituted benzene ring.

In another embodiment, X is N, CH or C—C≡N, W is an optionally substituted benzene ring, “U” is a fused optionally substituted benzene ring and R³ is hydrogen. In another embodiment, X is N, CH or C—C≡N, Y is NH, “U” is a fused optionally substituted benzene ring, and R³ is hydrogen.

In a further embodiment, X is N, CH or C—C≡N, Y is NH, “U” is a fused, optionally substituted benzene ring, W is an optionally substituted benzene ring and R³ is hydrogen.

Formula (II)

The following relate to possible embodiments of formula (II).

In one embodiment, in formula (II), A and B are each independently selected from C—R₁, C—R₂ and CH, and optionally at least one of A and B is C—R₁, C—R₂, and optionally A is C—R₁ and B is C—R₂.

In a further embodiment, A and B are each independently selected from C—R₁, C—R₂ and CH, and optionally at least one of A and B is C—R₁, C—R₂, and optionally A is C—R₁ and B is C—R₂ and X is N, CH or C—C≡N. In another embodiment, A and B are each independently selected from C—R₁, C—R₂ and CH, and optionally at least one of A and B is C—R₁, C—R₂, and optionally A is C—R₁ and B is C—R₂, and X is N, CH or C—C≡N and R³ is hydrogen. In another embodiment, A and B are each independently selected from C—R₁, C—R₂ and CH, and optionally at least one of A and B is C—R₁, C—R₂, and optionally A is C—R₁ and B is C—R₂, X is N, CH or C—C≡N and Y is NH. In another embodiment, A and B are each independently selected from C—R₁, C—R₂ and CH, and optionally at least one of A and B is C—R₁, C—R₂, and optionally A is C—R₁ and B is C—R₂, X is N, CH or C—C≡N and W is an optionally substituted benzene ring.

In a further embodiment, A and B are each independently selected from C—R₁, C—R₂ and CH, and optionally at least one of A and B is C—R₁, C—R₂, and optionally A is C—R₁ and B is C—R₂, X is N, CH or C—C≡N, Y is NH and W is an optionally substituted benzene ring.

In a further embodiment, A and B are each independently selected from C—R₁, C—R₂ and CH, and optionally at least one of A and B is C—R₁, C—R₂, and optionally A is C—R₁ and B is C—R₂, X is N, CH or C—C≡N, R³ is hydrogen, Y is NH and W is an optionally substituted benzene ring.

Formula (III)

The following relate to possible embodiments of formula (III).

In one embodiment in formula (III), X is N, CH or C—C≡N. In another embodiment, X is N, CH or C—C≡N and R³ is hydrogen. In another embodiment, X is N, CH or C—C≡N and Y is NH. In a further embodiment, X is N, CH or C—C≡N, R³ is hydrogen and Y is NH.

In one embodiment, the compound is N-[3-chloro-4-[(3-fluorophenyl)methoxy)phenyl]-6-[5-[(2-methylsulfonylethylamino)methyl]-2-furyl]quinazolin-4-amine, or any salt, base or prodrug form thereof.

In one embodiment, the compound is lapatinib, or any salt, base or prodrug form thereof.

In one embodiment, the compound is N-{3-chloro-4-fluoro-phenyl)-7-methoxy-6-(3-morpholin-4-ylpropoxy)quinazolin-4-amine, or any salt, base or prodrug form thereof.

In another embodiment, the compound is gefitinib, or any salt, base or prodrug form thereof.

In one embodiment, the compound is N-(3-ethynylphenyl)-6,7-bis(20methoxyethoxy)quinazolin-4-amine, or any salt, base or prodrug form thereof.

In another embodiment, the compound is erlotinib, or any salt, base or prodrug form thereof.

R¹ and R²

The following examples of R¹ and R² may apply to any aspect of any embodiment of the present invention, i.e. any of formulae (I), (II) and (III).

At least one of R¹ and R² may be an optionally substituted furan. Optionally, R¹ is an optionally substituted furan and R² is hydrogen, or R² is an optionally substituted furan and R¹ is hydrogen. The furan may be substituted by a (e.g. primary, secondary or tertiary) sulfonyl group and/or a (e.g. primary, secondary or tertiary) amino group. Alternatively, the furan may be substituted by a [(C₁₋₄)sulfonyl-(C₁₋₄)alkylamino]alkyl group.

R¹ or R² may be, for example, [(2-methylsulfonylethylamino)methyl]-2-furyl]. For example, R¹ may be [(2-methylsulfonylethylamino)methyl]-2-furyl] and R² may be hydrogen. Alternatively, R² may be [(2-methylsulfonylethylamino)methyl]-2-furyl] and R¹ may be hydrogen.

Alternatively, at least one of R¹ and R² may be an optionally substituted alkoxy group. The alkoxy group may be a C₁₋₈ alkoxy group, for example a C₁₋₄ alkoxy group. In an embodiment, the alkoxy group may be (S) or (R)-(tetrahydrofuran-3-yl)oxy. For example, R¹ or R² may be methoxy. The alkoxy group may alternatively be a morpholino-(C₁₋₄)alkoxy group. For example, R¹ or R² may be (3-morpholin-4-ylpropoxy). “Alkoxy” may be a di-alkoxy group.

One of R¹ and R² may be C₁₋₄ alkoxy, and the other may be morpholino-(C₁₋₄)alkoxy. For example, one of R¹ and R² may be methoxy and the other may be (3-morpholin-4-ylpropoxy). For example, R¹ may be (3-morpholin-4-ylpropoxy) and R² may be methoxy.

In an embodiment, at least one of R¹ and R² may be a di-alkoxy group. The di-alkoxy group may be, for example a di-C₁₋₈ alkoxy group, for example a di-C₁₋₄ alkoxy group. For example, at least one of R¹ and R² may be (2-methoxyethoxy). In another example, both R¹ and R² may be (2-methoxyethoxy) groups.

In an embodiment, at least one of R¹ and R² is (E)-dimethylamino(but-2-enamide). In an embodiment, one of R¹ and R² is (E)-dimethylamino(but-2-enamide) and the other of R¹ and R² is alkoxy, which is optionally selected from a C1 to 5 straight-chain unsubstituted-alkyloxy, optionally, methoxy or ethoxy, and (S) or (R)-(tetrahydrohydrofuran-3-yl)oxy. In an embodiment, R¹ is (E)-dimethylamino(but-2-enamide) and R² is alkoxy.

R₄ and R₅

The following examples of R⁴ and R⁵ may apply to any aspect of any embodiment of the present invention, i.e. any of formulae (I), (II) and (III).

At least one of R⁴ and R⁵ may a halogen. The halogen may be, for example, fluorine, chlorine, bromine or iodine.

Both R⁴ and R⁵ may be a halogen, for example. For example, one of R⁴ and R⁵ may be fluorine and the other may be chlorine. For example, in formula (III), R⁴ may be chlorine located at the 3-position on the benzene ring and R⁵ may be fluorine located at the 4-position on the benzene ring (where Y is at the 1-position on the benzene ring).

Optionally, at least one of R⁴ and R⁵ is an alkoxy group. The alkoxy group may be an optionally substituted aryl or heteroaryl alkoxy group, wherein optionally the optionally substituted aryl or heteroaryl alkoxy group is selected from optionally substituted phenyl alkoxy group and optionally substituted pyridinyl alkoxy group, for example, optionally substituted pyridine-2-yl alkoxy, for example pyridine-2-yl methoxy. Optionally, the alkoxy group may be a halogen-substituted phenyl alkoxy group. The alkoxy group may be, for example, (3-fluorophenyl)methoxy.

In one example, R⁴ may be a halogen and R⁵ may be an alkoxy group. For example, R⁴ may be a halogen and R⁵ may be an optionally substituted phenyl alkoxy group or pyridinyl alkoxy group. In one example, R⁴ is chloro and R⁵ is (3-fluorophenyl)methoxy or pyridine-2-yl methoxy, and optionally, in formula (III), R⁴ may be located at the 3-position on the benzene ring and R⁵ may be located at the 4-position on the benzene ring (where Y is at the 1-position on the benzene ring).

Alternatively, at least one of R⁴ and R⁵ may be an alkynyl group. The alkynyl group may be, for example, a C₂₋₈ alkynyl group, for example a C₂₋₄ alkynyl group. In one embodiment, at least one of R⁴ and R⁵ is ethynyl. In one embodiment, R⁴ is ethynyl located at the 3-position on the benzene ring and R⁵ is hydrogen.

Combination of R¹, R², R⁴ and R⁵

The following combinations of R¹, R², R⁴ and R⁵ may apply to any aspect of any embodiment of the present invention, i.e. any of formulae (I), (II) and (III).

R¹ may be an optionally substituted furan, R² may be hydrogen, R⁴ may be a halogen and R⁵ may be an alkoxy. For example, R¹ may be a furan substituted by a [(C₁₋₄)sulfonyl-(C₁₋₄)alkylamino]alkyl group, R² may be hydrogen, R⁴ may be a halogen and R⁵ may be a optionally substituted phenyl alkoxy group, for example a halogen substituted phenyl alkoxy group.

For example, R¹ may be [(2-methylsulfonylethylamino)methyl]-2-furyl], R² may be hydrogen, R⁴ may be chloro and R⁵ may be (3-fluorophenyl)methoxy.

In an embodiment, R¹ and R² may be alkoxy groups and R⁴ and R⁵ may be halogen. For example, R¹ may be a C₁₋₄ alkoxy group, R² may be a morpholino-(C₁₋₄)alkoxy group and R⁴ and R⁵ may be halogen.

For example, R¹ may be methoxy, R² may be (3-morpholin-4-ylpropoxy), R⁴ may be chloro and R⁵ may be fluro.

Alternatively, R¹ and R² may be alkoxy groups, R⁴ may be an alkynyl group and R⁵ may be hydrogen. For example, R¹ and R² may be di-(C₁₋₈) alkoxy groups and R⁴ may be a C₂₋₈ alkynyl group. For example, R¹ and R² may be di-(C₁₋₄) alkoxy groups and R⁴ may be a C₂₋₄ alkynyl group.

For example, R¹ and R² may be (2-methoxyethoxy), R⁴ may be (3-ethynyl) and R⁵ may be hydrogen.

In an embodiment, one of R¹ and R² is (E)-dimethylamino(but-2-enamide) and the other of R¹ and R² is alkoxy, which is optionally selected from a C1 to 5 straight-chain unsubstituted-alkyloxy, optionally, methoxy or ethoxy, and (S) or (R)-(tetrahydrohydrofuran-3-yl)oxy, and at least one of R⁴ and R⁵ is halogen. In an embodiment, one of R¹ is (E)-dimethylamino(but-2-enamide) and R² is alkoxy, which is optionally selected from a C1 to 5 straight-chain unsubstituted-alkyloxy, optionally, methoxy or ethoxy, and (S) or (R)-(tetrahydrohydrofuran-3-yl)oxy, R⁴ is halogen, preferably CI, and R⁵ is selected from halogen and a pyridinyl alkoxy group. In one example, R⁴ is chloro and R⁵ is (3-fluorophenyl)methoxy or pyridine-2-yl methoxy. In an embodiment, R¹ is (E)-dimethylamino(but-2-enamide) and R² is ethoxy or)-(tetrahydrohydrofuran-3-yl)oxy, R⁴ is chloro and R⁵ is selected from fluorine and pyridine-2-yl methoxy, and optionally in formula (III), R⁴ is located at the 3-position on the benzene ring and R⁵ is located at the 4-position on the benzene ring (where Y is at the 1-position on the benzene ring).

Optionally, in any of the formulae, R¹ and R² are 2-methoxyethoxy.

Optionally, the therapeutic agent comprises N-[3-chloro-4-[(3-fluorophenyl)methoxy]phenyl]-6-[5-[(2-methylsulfonylethylamino)methyl]-2-furyl]quinazolin-4-amine. Optionally, the agent comprises lapatinib.

Optionally, the therapeutic agent comprises N-(3-chloro-4-fluoro-phenyl)-7-methoxy-6-(3-morpholin-4-ylpropoxy)quinazolin-4-amine. Optionally, the agent comprises gefitinib.

Optionally, the therapeutic agent comprises N-(3-ethynylphenyl)-6,7-bis(2-methoxyethoxy)quinazolin-4-amine. Optionally, the agent comprises erlotinib.

Optionally, the therapeutic agent comprises (2E)-N-[4-[[3-chloro-4-[(pyridin-2-yl)methoxy]phenyl]amino]-3-cyano-7-ethoxyquinolin-6-yl]-4-(dimethylamino)but-2-enamide. Optionally, the agent comprises neratinib.

Optionally, the therapeutic agent comprises N-[4-[(3-Chloro-4-fluorophenyl)amino]-7-[[(3S)-tetrahydro-3-furanyl]oxy]-6-quinazolinyl]-4(dimethylamino)-2-butenamide. Optionally, the therapeutic agent comprises (E)-4-Dimethylamino-but-2-enoic acid{4-(3-chloro-4-fluoro-phenylamino)-7-[(S)-(tetrahydro-furan-3-yl)oxy]-quinazolin-6-yl}-amide. Optionally, the therapeutic agent comprises afatinib.

Optionally, the therapeutic agent comprises an antibody selected from cetuximab, trastuzumab, pertuzumab, peruzumab, and panitumumab.

In an embodiment, the subject is suffering from thymic atrophy and/or involution and another disorder, and the therapeutic agent is for treating or treats the other disorder.

Optionally, the other disorder is selected from a viral infection and a bacterial infection. Optionally, the other disorder is a bacterial infection selected from bacterial pneumonia, methicillin-resistant Staphylococcus aureus (MRSA), Clostridium difficile and vancomycin-resistant enterococcus (VRE).

Optionally, the other disorder is a viral infection selected from influenza, respiratory syncytial virus (RSV) and a herpes virus such as herpes zoster.

Optionally, the other disorder is selected from HIV and AIDS.

Optionally the other disorder is selected from XLAAD (X-linked autoimmunity and allergic dysregulation; sometimes termed IPEX (immune dysfunction, polyendocrinopathy, and enteropathy, X-linked)), APECED (Autoimmune polyendocrine syndrome type 1), DiGeorge syndrome and Systemic Lupus Erythematosus.

Optionally, the subject is receiving a vaccine for the other disorder.

Optionally, the subject is immunocompromised. The subject may be immunocompromised if the subject has experienced a loss, e.g. a significant loss, of thymus mass on CT or MRI scan and/or reduced peripheral WBC abundance and/or reduced responsiveness to vaccination.

Optionally, the subject is malnourished. Optionally, the subject is receiving or has received, and optionally is recovering from, chemo- and/or radiotherapy.

Optionally, the subject is a human of 50 years of age or more. Optionally, the subject is a human of 60 years of age or more. Optionally, the subject is a human of 65 years of age or more. Optionally, the subject is a human of 70 years of age or more.

As described, above, in an aspect, the present invention provides a composition comprising the therapeutic agent as described herein. The composition may comprise the therapeutic agent and a pharmaceutical acceptable carrier or excipient, and the composition may be for use in a method for modulating the function and/or growth of a thymus in a subject, wherein the therapeutic agent comprises an HER2 or HER1 pathway antagonist or agonist, the method involving administering the therapeutic agent to the subject. The composition may be for treating thymic atrophy and/or involution in a subject, and the therapeutic agent may be or comprise an HER2 or HER1 pathway antagonist. The composition may be for treating a hyperactive thymus and/or excessive thymic growth in a subject, and the therapeutic agent may be a HER2 or HER1 pathway agonist and/or a CCR/CCL5 antagonist. The composition may be for treating a disorder in a subject, the disorder selected from systemic autoimmunity, peripheral autoimmunity and Systemic Lupus Erythematosus.

As described above, in an aspect, the present invention provides a method for modulating the function and/or growth of a thymus in a subject, the method involving administering a therapeutic agent or the pharmaceutical composition described herein to the subject, wherein the therapeutic agent comprises an HER2 or HER1 pathway antagonist or agonist. In an embodiment, the method is for treating thymic atrophy and/or involution in the subject, and wherein the agent comprises an HER2 or HER1 pathway antagonist. In an embodiment, the method is for treating a hyperactive thymus and/or excessive thymic growth in a subject, and the therapeutic agent is or comprises a HER2 or HER1 pathway agonist and/or a CCR/CCL5 antagonist.

The administration of the therapeutic agent can be effected by any method which enables delivery of the therapeutic agent to the required site, e.g. the thymus. These methods include, but are not limited to, oral routes, intraduodenal routes, parenteral injection (including intravenous, subcutaneous, intramuscular, intravascular or infusion) and topical administration.

The amount of therapeutic administered will, of course, be dependent on the subject being treated, on the severity of the affliction, on the manner of administration and on the judgment of the prescribing physician. However an effective dosage may be in the range of approximately 0.001-100 mg/kg, preferably 1 to 35 mg/kg in single or divided doses. For an average 70 kg human, this would amount to 0.05 to 7 g/day, preferably 0.2 to 2.5 g/day.

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition may include a conventional pharmaceutical carrier or excipient and the therapeutic agent as described herein. In addition, it may include other medicinal or pharmaceutical agents, carriers, adjuvants, etc.

Pharmaceutical compositions according to the invention may contain 0.1%-95% of the therapeutic agent, preferably 1%-70%. In any event, the composition or formulation to be administered will contain a quantity of therapeutic agent in an amount effective to alleviate or reduce the signs in the subject being treated, over the course of the treatment.

Exemplary parenteral administration forms include solutions or suspensions of the therapeutic agent in sterile aqueous solutions, for example aqueous propylene glycol or dextrose solutions. Such dosage forms can be suitably buffered, if desired. Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Suitable materials, therefore, include lactose or milk sugar and high molecular weight polyethylene glycols. When aqueous suspensions or elixirs are desired for oral administration, the therapeutic agent therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of the therapeutic agent are known, or will be apparent, to those skilled in this art. For examples, see Remington's Pharmaceutical Sciences., Mack Publishing Company, Easter, Pa., 15th Edition (1975).

The therapeutic agent described above may be applied as a sole therapy or may involve, in addition to the therapeutic agent, one or more other active substances may be administered, including, but not limited to, an anti-bacterial agent, an anti-viral agent, and a vaccine for a disorder, for example as described above. Such conjoint treatment may be achieved by way of the simultaneous, sequential, cyclic or separate dosing of the individual components of the treatment.

The present invention provides prodrugs of the compounds, which may be of formula (I). As will be understood by the skilled person, some of the compounds useful for the methods of the present invention may be available as prodrugs. As used herein, the term “prodrug” refers to a compound of formula (I) which has been structurally modified such that in vivo the prodrug is converted, for example, by hydrolytic, oxidative, reductive, or enzymatic cleavage, into the parent molecule (“drug”) as given by formula (I). Such prodrugs may be, for example, metabolically labile ester derivatives of the parent compound where said parent molecule bears a carboxylic acid group. Conventional procedures for the selection and preparation of suitable prodrugs are well known to one of ordinary skill in the art.

The present invention provides pharmaceutically acceptable salts of the compounds, which may be of formula (I). Pharmaceutically acceptable salts include addition salts, including salts formed with acids or bases. The acids may be selected from inorganic acids, for example hydrochloric, hydrobromic, nitric, sulphuric or phosphoric acids, phosphonic, or with organic acids, such as organic carboxylic acids, for example acetic, trifluoroacetic, lactic, succinic, glutaric, ascorbic, pyruvic, lactobionic, glycolic, oxalic, maleic, hydroxymaleic, fumaric, malic, malonic, tartaric, citric, salicylic, o-acetoxybenzoic, or organic sulphonic, 2-hydroxyethane sulphonic, toluene-p-sulphonic, methanesulphonic, camphoric, bisethanesulphonic acid or methanesulphonic acid. The bases may be selected from sodium hydroxide, potassium hydroxide, triethylamine, tert-butylamine.

In an aspect, the present invention provides a therapeutic agent for use in a method for treating a disorder in a subject, the disorder selected from systemic autoimmunity, peripheral autoimmunity and Systemic Lupus Erythematosus, wherein the therapeutic agent is as described herein, for example is or comprises an HER2 or HER1 pathway antagonist or agonist the method involving administering the therapeutic agent to the subject. The HER2 or HER1 pathway antagonist or agonist may be as defined herein. Peripheral autoimmunity may be defined as ‘inflammation and auto reactive leukocyte infiltration to peripheral organs such as the skin’. Systemic autoimmunity may be defined as ‘inflammation and auto reactive leukocyte infiltration to internal organs and tissues’. The systemic autoimmunity may be selected from systemic lupus erythematosus, IPEX/XLAAD syndrome, myasthenia gravis, and APECED syndrome The peripheral autoimmunity may be selected from systemic lupus erythematosus, psoriasis, or autoimmune alopecia. The subject having the disorder selected from systemic autoimmunity, peripheral autoimmunity and Systemic Lupus Erythematosus may or may not be suffering from any of thymic atrophy, thymic involution, a hyperactive thymus and excessive thymic growth. The therapeutic agent for the aspect in this paragraph may be any therapeutic agent described herein, and a composition comprising said therapeutic agent may be as described herein. The therapeutic agent may be administered as described herein. The subject to which the therapeutic agent is administered for the aspect in this paragraph may be as described herein.

It should be understood that the invention is not limited to the particular embodiments shown and described herein, but that various changes and modifications may be made without departing from the spirit and scope of the invention as defined by the claims. Embodiments of the invention are described below with reference to the following non-limiting Examples and accompanying figures.

### EXAMPLES

### Example 1

Methods

Animal Husbandry

Bitransgenic mice exhibiting a Keratin 14-expressing cell specific, doxycycline-inducible, mutant Erbb2 (Her2) transgene (termed BiTg mice) were produced by breeding commercially available heterozygous K14-rtTA (#008099, Jackson Laboratory, USA) and heterozygous tetO-Erbb2 (#010577, Jackson Laboratory, Bar Harbour, USA) mouse strains. BiTg offspring were produced at expected Mendelian ratios (25% of offspring) and identified by genomic PCR screening. All BiTg mice and controls were used at between 8-12 weeks of age.

PCR genotyping was performed using the following primers:

tetO-Erbb2-forward primer
[Seq. ID 1]
agcagagctcgtttagtg
tetO-Erbb2-reverse primer
[Seq. ID 2]
ggaggcggcgacattgtc
K14-rtTA-forward primer
[Seq. ID 3]
cacgatacacctgactagctgggtg
K14-rtTA-reverse primer
[Seq. ID 4]
catcacccacaggctagcgccaact

Eighteen month old C57/BI6N wildtype mice used in Lapatinib studies were purchased from Charles River Laboratories (UK). All mice were housed on a 12 hour light/dark cycle, in individually ventilated cages with access to standard rodent diet (Harlan, UK) and tap water ad libitum. Mice were euthanized by intraperitoneal injection of 0.2 mL sodium pentobarbital (Euthatal, Merial Animal Health, UK).

In Vivo Experimental Procedures

Doxycycline water (Dox) was prepared by dissolving 500 mg doxycycline (#D9891, Sigma, UK) and 5 g sucrose (#S/8560/60, Fisher, UK) in 250 mL water. Dox was administered ad libitum in a foil-wrapped standard drinking bottle; fresh Dox was given every 48 hours. 300 mg Lapatinib (#J62401, Alfa Aesar, USA) was prepared in 12 mL DMSO (#472301, Sigma, UK) at a final concentration of 25 mg/mL. Mice were given 2.5 mg (100□L) Lapatinib or an equivalent dose of DMSO alone 5 times per week for 4 weeks by oral gavage. One hour prior to euthanasia all mice for subsequent histological analysis were injected with 150 μL of 10 mg/mL BrdU solution (Invitrogen, UK) to label proliferating cells.

Tissue Preparation and Histology

Tissue was prepared for histological analysis by careful microdissection to remove the heart, lungs, and extrathymic connective tissue followed by overnight fixation in 10% neutral buffered formalin (Sigma, UK) at 4 degrees Celsius. Tissue was processed through graded 70% ethanol, xylene, and paraffin washes for wax embedding using an automated tissue processor (TP1050, Leica, Germany). Adjacent serial sections of thymus were cut at 5 mm using a microtome, mounted on glass microscope slides (Superfrost plus, VWR, UK), and stained with hematoxylin and eosin using an automated staining system (TissueTek, Germany) or left unstained for subsequent immunohistochemistry.

Thymus and lymph nodes were prepared for flow cytometry by incubation in a 2 mL solution of RPMI 1650 (Invitrogen, UK) containing Liberase TL (0.3 mg/mL; Roche, Germany) plus DNAse I (0.2 mg/ml, Roche, Germany) for 30 minutes at 37 degrees Celsius. Thymus and lymph node tissue was then macerated through a 70 μM filter (BD, USA) and enzymes inactivated by further washing with 30 mL PBS (Invitrogen, UK) plus 5 mM EDTA (Sigma, UK). This single cell suspension was centrifuged for 5 minutes at 1250 rpm, the supernatant removed, and the cell pellet resuspended in 3 mL RPMI 1640 (lymph nodes) or 5 mL RPMI 1640 (thymus). Total cell counts were obtained using a Millicell automated hematocytometer. Blood cells were prepared for flow cytometry by direct blood collection from the descending aorta in a capillary Microvette tube containing EDTA (CB 300 K2E, Sarstedt, Germany), elimination of red blood cells using lysis buffer (R7757, Sigma, UK) and dilution in 30 mL RPMI 1640. Blood cells were then pelleted by centrifugation as above and resuspended in 20 mL RPMI 1640 and cell counts obtained using a Millicell automated hematocytometer.

Flow Cytometry and Immunohistochemistry

All cell suspensions (thymus, lymph node, blood) were diluted to 1 million cells/mL for antibody staining and flow cytometry analysis. Antibodies for flow cytometry analysis were as follows: CD3-FITC (17a2, Invitrogen, UK), CD4-PE (I3t4, BD Pharmingen, UK), CD8a-Q605 (53-6.7, eBioscience, UK), CD19-A421 (6D5, Biolegend, UK), CD11b-A421 (eBioscience, UK), Ter119-A421 (TER119, eBioscience, UK), CD47-APC780 (1M7, eBioscience, UK), CD25-A660 (7D4, eBioscience, UK), Nk1.1-APC780 (PK136, eBioscience, UK), NkP46-a660 (29a1.4, eBioscience, UK). All antibodies were diluted to 2 μL/10̂6 cells in 1 mL final volume; unstained and single antibody stained controls were used to set all voltage and gate compensations. Antibody staining was for 30 minutes at 4 degrees Celsius, after which time cells were centrifuged at 1250 rpm for 5 minutes and cell pellets resuspended in 1 mL RPMI 1640. All flow cytometry analysis was performed on either an LSR2 or LSR Fortessa flow cytometry system. A minimum of 10,000 events was collected for each analysis (phenotype, T cell developmental stage, peripheral blood and lymph node cell analysis). All experiments represent at least an n=4 for each condition and all analyses were done using FlowJo software. Thymus tissue sections were prepared for BrdU immunohistochemistry by dewaxing using an automated tissue staining system (TissueTek), blocking for 1 hour at room temperature in blocking solution (PBS containing 10% fetal bovine serum (10270-106, Invitrogen, UK) plus fish skin gelatin (G7765, Sigma, UK)). A BrdU primary antibody (rat, Covance, UK) was diluted in blocking solution and slides were incubated overnight in this antibody solution at 4 degrees Celsius. Slides were then washed in PBS and an anti-rat Alexa 555 secondary antibody (Invitrogen, UK) was applied in blocking solution for 3 hours at room temperature, washed, and slides coverslipped using Mowiol mounting media.

Magnetic Cell Separation

Magnetic cell separation for subsequent thymus stromal cell proteome profiling was performed using an AutoMACS cell separator (Miltenyi, Germany). Briefly, single thymus cell suspensions were prepared as described above and suspended in 80 μL MACS staining buffer (Miltenyi, Germany). Ten μL of both CD31 and CD45 microbeads (130-097-418 and 130-052-301, Miltenyi, Germany) were incubated with 10̂7 total thymocytes for 15 minutes at 4 degrees Celsius, washed, and resuspended in 1 mL MACS staining buffer. CD45 and CD31 negative stromal thymus cells were then purified using the ‘deplete_s’ standard MACS protocol and the purity of this depletion confirmed by flow cytometry (as described above). A minimum 12-fold enrichment in CD31 and CD45-negative thymocytes was achieved for all samples.

Imaging

Gross pathological images of thymus organs were obtained using a digital SLR camera (Nikon, USA). Images of hematoxylin and eosin stained thymus tissue sections were obtained using a Nanozoom automated slide scanning system (Hammatsu, Japan).

Images of BrdU stained sections were obtained using a Zeiss Axioscope microscope with epifluorescence and digitial imaging (Zeiss, Germany). All images were cropped and adjusted for brightness and contrast in Adobe photoshop and all final figures were prepared using Adobe Illustrator (Adobe, USA).

Protein and Gene Expression Assays

We interrogated the publicly available Immgen immunological genome database (http://www.immgen.org) to assess the relative expression levels of Her1 and Her2 in thymus stroma and immune cell populations. We used a Proteome Profiler Mouse Cytokine Array Panel A for all protein analyses (ARY006; R&D Systems, USA). Briefly, protein samples were prepared by lysing pelleted CD45/CD31 depleted thymus stroma samples in 100 μL RIPA buffer with complete MINI protease inhibitors (Roche, USA). We determined total protein content in all RIPA samples using a commercially available BCA assay (Promega, USA) and loaded 100 μg total protein on to each blot. Samples were normalised for protein loading based on the blot internal control (reference spot).

Statistical Analysis

All graphs were prepared in Microsoft Excel and GraphPad Prism using total cell abundance, organ weight, and/or abundance of different cell phenotypes identified by flow cytometry. All error bars represent the standard error of the mean; statistical significance was accepted at p<0.05 (*), p<0.005 (**) or p<0.005 (***). We used a Student's t-test for pairwise comparisons among samples.

Results

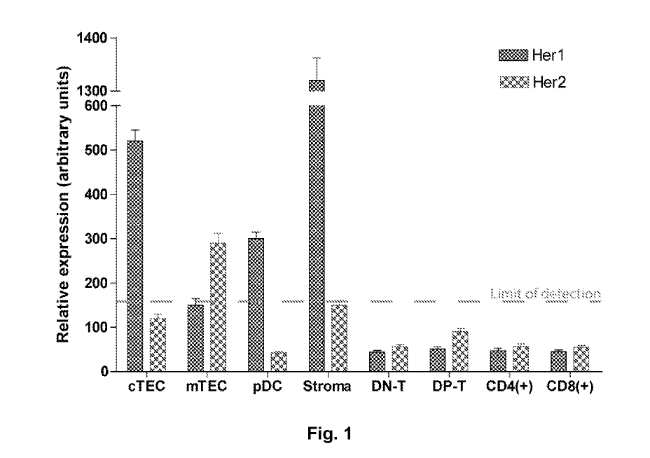

FIG. 1 illustrates that cortical and medullary thymus epithelial cells express Her1 and Her2. The present inventors used the publicly available IMMGEN database to query Her1 and Her2 expression in murine thymus stroma and immune cell populations. Her1 was expressed in cortical thymus epithelial cells (cTEC), plasmacytoid dendritic cells (pDC) and thymus stroma populations whereas Her2 was present only in medullary thymus epithelial cells (mTEC). No immune cell populations (double negative T (DN-T), double positive T (DP-T), CD4 or CD8) expressed detectable levels of Her1 and Her2.

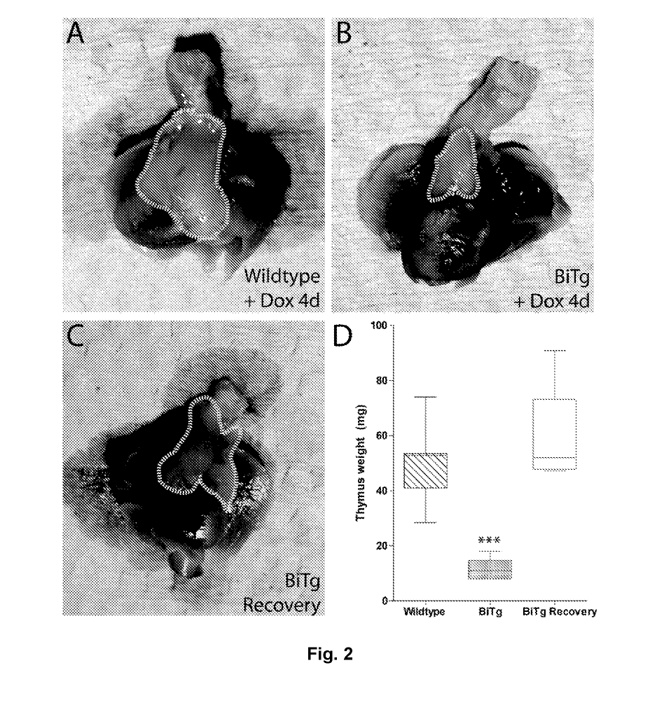

FIG. 2 illustrates that thymic epithelial Her2 activation caused rapid onset, reversible thymus involution. BiTg mice expressing Dox-inducible ERbb2 were treated with doxycycline for 4 days via their drinking water. Mice were sacrificed at this time or allowed to recover for a further 28 days after withdrawal of Dox (BiTg Recovery). Thymus weights were compared with single transgenic (K14-rtTA and tetO-Erbb2) and wildtype animals treated with Dox for an equivalent period of time. (A-C) Photographs of gross thymus appearance in wildtype, BiTg, and BiTg recovery mice treated with Dox. The dashed line demarcates the thymus organ. (D) Thymus weights in wildtype, BiTg, and BiTg recovery animals. (n=5-8 mice per group; asterisks indicate significance at p<0.0005)

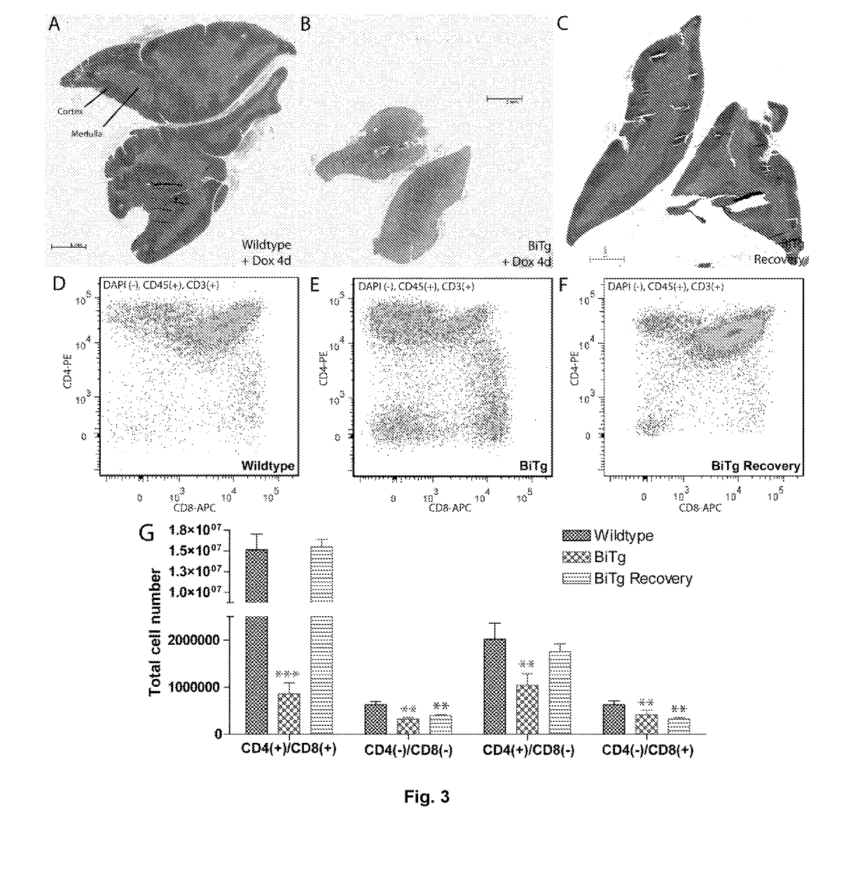

FIG. 3 illustrates that Her2 activation causes depletion of thymic cortex and immature (DP-T) T lymphocytes. (A-C) Tissue sections of wildtype (A), BiTg (B) and BiTg recovery (C) thymuses were compared using hematoxylin and eosin staining. Lines indicate regions of thymus cortex and medulla identifiable by staining intensity. (D-F) Flow cytometry plots of wildtype (D), BiTg (E), and BiTg recovery (F)DAPI(−), CD45(+) thymus cell populations stained with CD4 (y-axis) and CD8 (x axis). (G) Quantification of DN-T, DP-T, CD4 and CD8 single T cell populations present in wildtype, BiTg and BiTg recovery thymuses (n=5-8 per group; asterisks denote significance at p<0.05 (*), p<0.005 (**) or p<0.0005 (***).

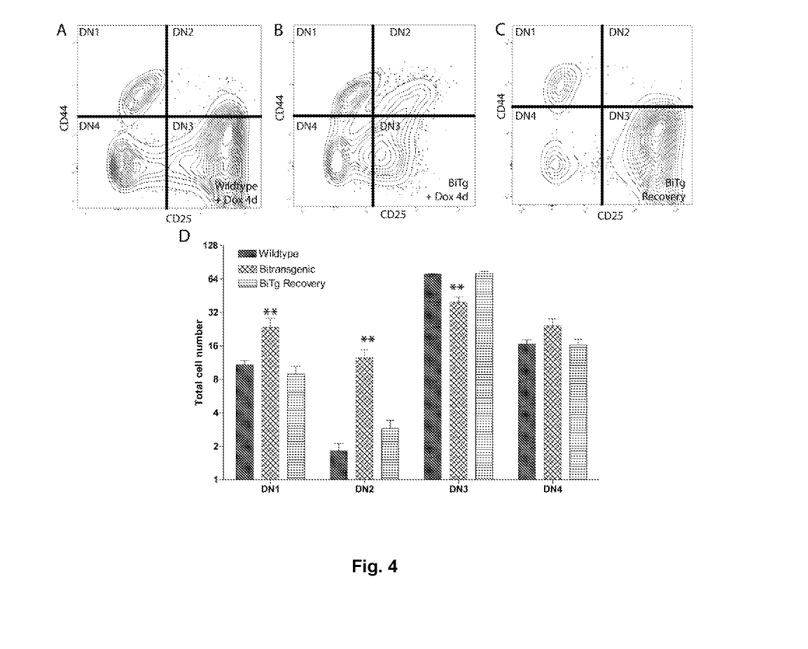

FIG. 4 illustrates that thymic epithelial Her2 activation alters T lymphocyte development. (A-C) The present inventors assessed immature T lymphocyte development in wildtype, BiTg, and BiTg recovery mice by examining CD44 and CD25 expression in CD4/CD8/lineage negative thymocytes using flow cytometry. Immature T lymphocytes were classified as DN1 (CD44+/CD25−), DN2 (CD44+/CD25+), DN3 (CD44−/CD25+), or DN4 (CD44−CD25−) on this basis. (D) Quantification of DN1-4 abundance in all treatment groups revealed that activation of Her2 in BiTg significantly increased DN1 and DN2 thymocyte abundance and reduced DN3 cell numbers. All of these Her2-dependent changes were entirely reversible following withdrawal of Her2 activation (BiTg recovery). We used a minimum n=3 for all treatment conditions. Asterisks (**) denote significance at p<0.005.

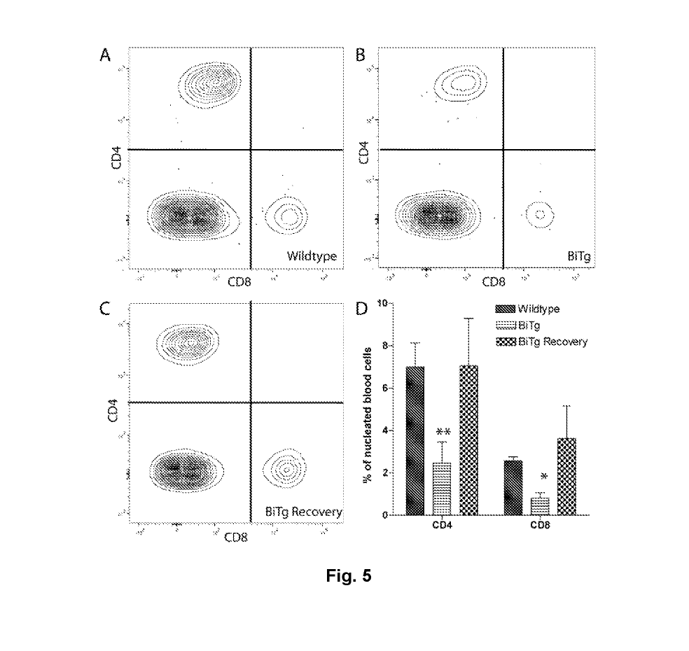

FIG. 5 illustrates that thymic epithelial Her2 activation causes depletion of T lymphocytes from peripheral blood. (A-C) The present inventors used flow cytometry antibody staining for CD4 and CD8 to assess mature T lymphocyte abundance in the peripheral blood of wildtype, BiTg and BiTg recovery animals. (D) Quantification of CD4 and CD8 cell abundance as a percentage of all nucleated blood cells revealed that activation of Her2 in the thymus epithelium reduced both CD4 and CD8 cell abundance in peripheral blood. This effect was entirely reversible following reversal of Her2 activation (BiTg recovery). The present inventors used a minimum n=5 for all treatment conditions.

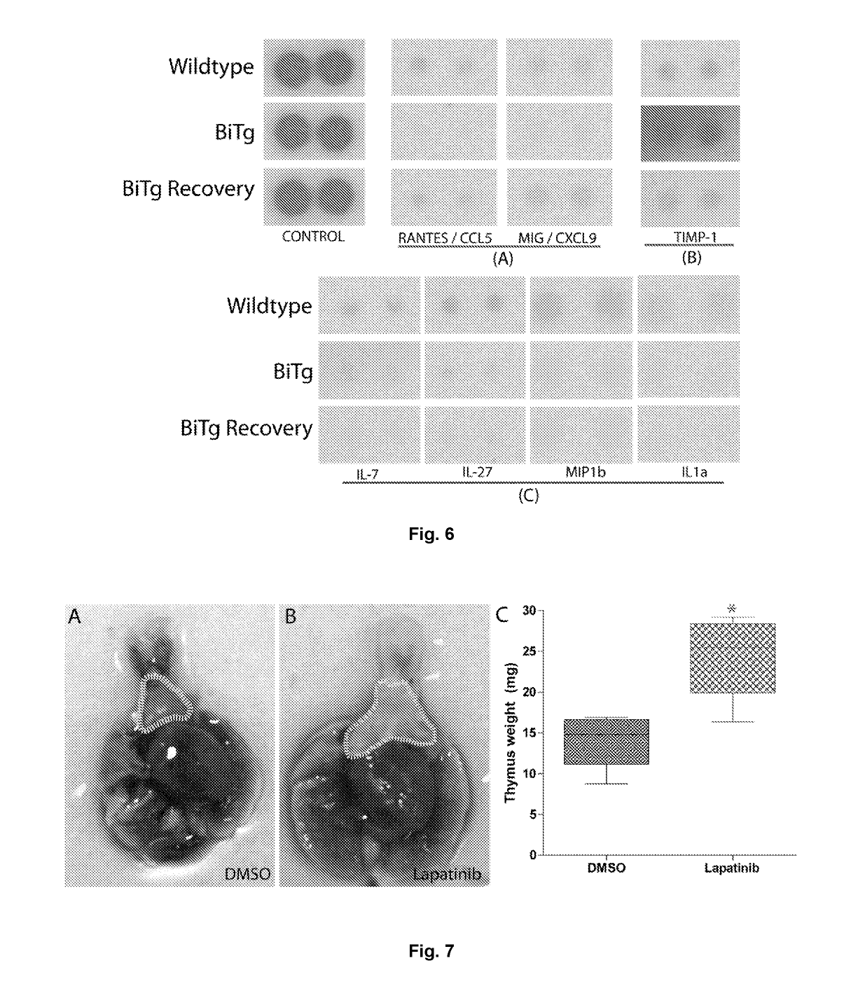

FIG. 6 illustrates that thymic epithelial Her2 activation alters thymic stromal cell cytokine abundance. Non-hematopoietic/non-endothelial thymus stromal cells were enriched using magnetic antibody bead separation. A minimum 13-fold enrichment was achieved for all samples from which protein samples were purified and applied to a commercially available murine cytokine proteome profiler assay. The present inventors found that both RANTES/CCL5 and MiG/CXCL9 were dynamically regulated (reduced upon Her2 activation then restored during recovery) in response to thymus Her2 activation. We also found that IL7, IL27, IL-1a, and MIP1beta were reduced but not restored following Her2 activation and recovery. In addition they also found that TIMP1 was transiently increased upon Her2 activation.

FIG. 7 illustrates that treatment with lapatinib increases thymus size in elderly animals. Geriatric (18 month old) C57/BI6N wildtype mice were randomized to control and treatment groups and administered 12.5 mg lapatinib in 10% DMSO per week for 5 weeks by oral gavage (2.5 mg/day; 5× treatments/week). Control mice received an equivalent dose of 10% DMSO alone. (A, B) Gross appearance of DMSO (A) and Lapatinib-treated (B) thymus organs (outlined). (C) Quantification of thymus weight after 5 weeks control or Lapatinib treatment (n=10/treatment group; asterisk indicates significance at p<0.05).

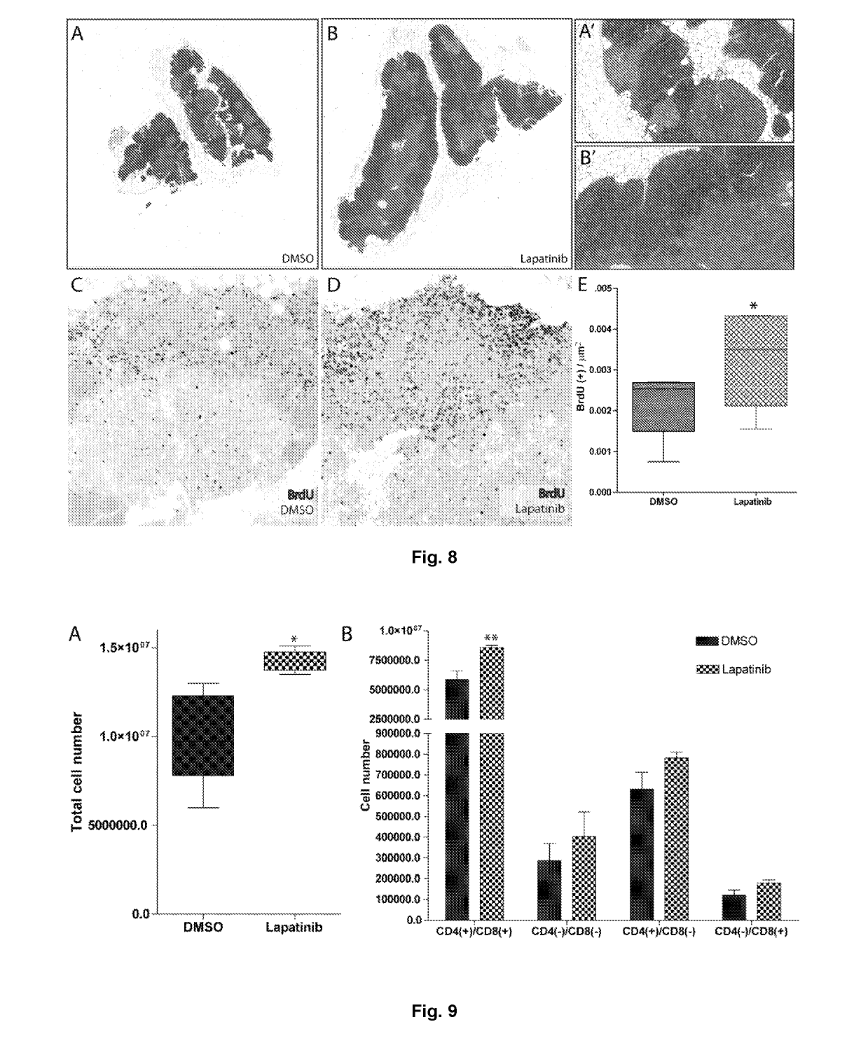

FIG. 8 illustrates that lapatinib exposure reverses thymus involution in elderly animals. (A, B) Lapatinib treatment restored thymic cortico-medullary boundaries and improved overall thymus histological appearance. (C, D) Adjacent control and Lapatinib treated thymus sections were stained with BrdU to assess thymus cell proliferation (black stain). (E) Quantification of BrdU cell abundance in control and Lapatinib treated thymus reveals a statistically significant increase in thymocyte proliferation following Lapatinib exposure (n=5/treatment type; p<0.05).

FIG. 9 illustrates that lapatinib increases thymus immature T lymphocyte production in elderly individuals. (A) Lapatinib exposure to 15-18 month old mice increased thymus cell abundance when compared with DMSO treated controls. (B) Lapatinib administration increases production of immature (double positive, DP-T) T lymphocytes when compared with DMSO-treated controls. The present inventors also observed a trend towards increased CD4, CD8, and DN-T T cell production in Lapatinib treated mice compared with DMSO treated controls (n=5 mice per treatment group).

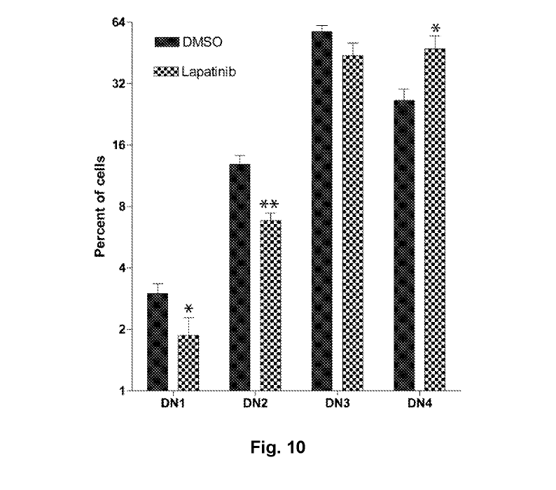

FIG. 10 illustrates that lapatinib exposure affects thymus T lymphocyte development. We assessed immature T lymphocyte development in DMSO and Lapatinib treated 15 month old mice by examining CD44 and CD25 expression in CD4/CD8/lineage negative thymocytes using flow cytometry. Lapatinib exposure significantly reduced DN1 (CD44+/CD25−) and DN2 (CD44+/CD25+) immature T cell abundance and increased DN4 (CD44/CD25 negative) cell abundance. (n=5 mice/treatment group).

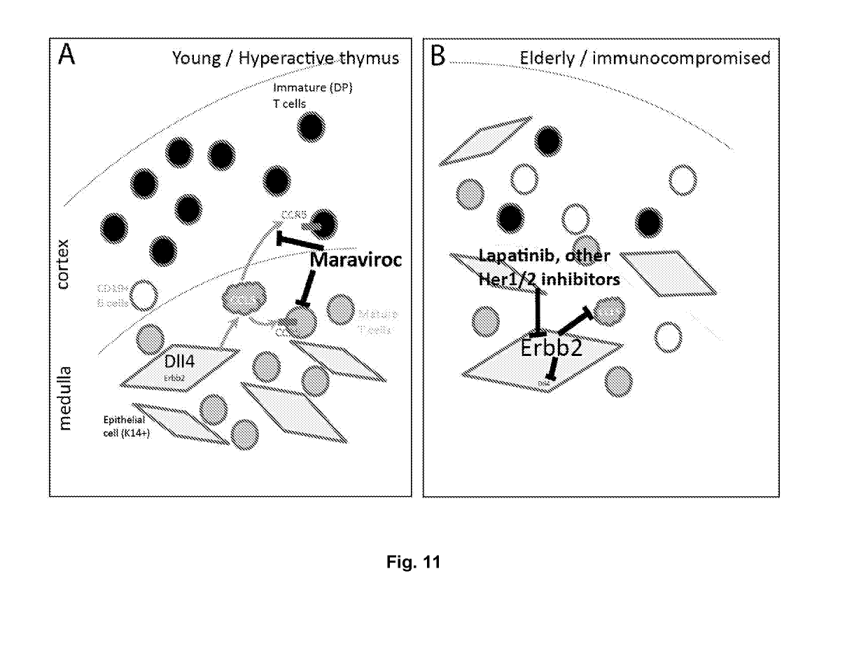

FIG. 11 illustrates a model depicting pharmacological modulators of thymus function in elderly and immunocompromised individuals and their potential mechanisms of action.

### Example 2

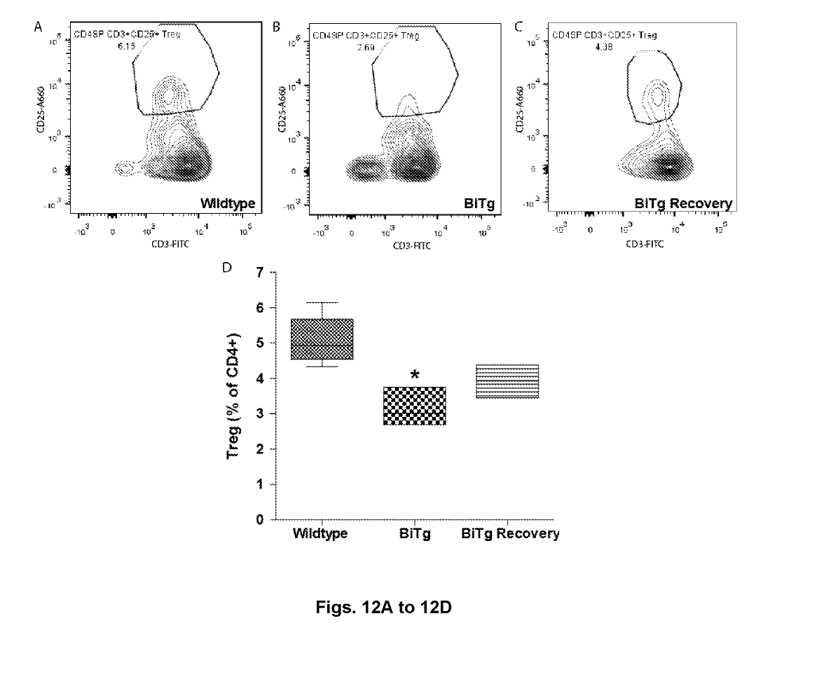

FIG. 12 illustrates that experimentally induced HER2 activation causes reversible depletion of regulatory T cell (Treg, CD4SP, CD3+CD25+) populations from the thymus. (A-C) Representative images of Treg abundance in wildtype (A) and Bitransgenic (B) mice treated with doxycycline for 3 days or bitransgenic mice treated with doxycycline for 3 days and allowed to recover for 28 days (BiTg recovery). (D) Quantification of Treg abundance in all treatment groups revealed that activation of Her2 in bitransgenic mice significantly reduced Treg cell abundance. This Her2-dependent change was partially reversible upon withdrawal of Her2 activation. The present inventors used a minimum n=3 animals for all treatment conditions. Asterisk (*, D) denotes significance at p<0.05.

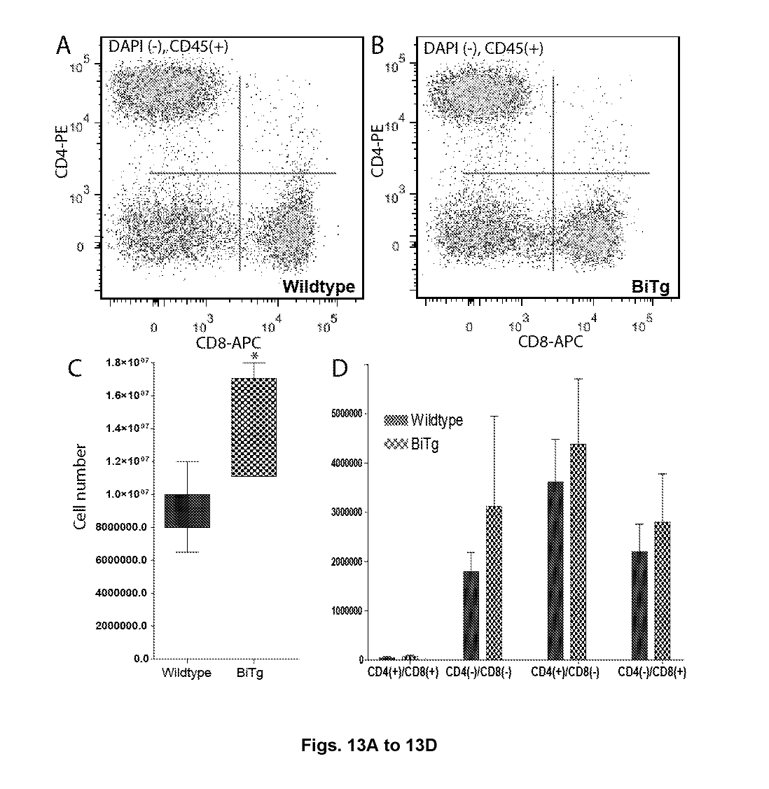

FIG. 13 illustrates that thymic epithelial Her2 activation causes an increase in peripheral lymph node cellularity but does not alter lymph node T cell phenotypes. (A, B) The present inventors assessed T cell abundance in wildtype and bitransgenic lymph nodes by examining CD4 and CD8 expression using flow cytometry. T cell phenotypes present were either CD4 or CD8 single positive mature T cells. (C) Total cellularity of lymph nodes was increased in bitransgenic mice as determined by cell counting. (D) Quantification of T cell phenotypes in wildtype and bitransgenic lymph nodes. Overall, this data confirms that peripheral autoimmunity and increased lymph node cellularity is a component of thymic epithelial Her2 activation. The present inventors used a minimum n=3 animals for all treatment conditions. Asterisk (*, C) denotes significance at p<0.05.

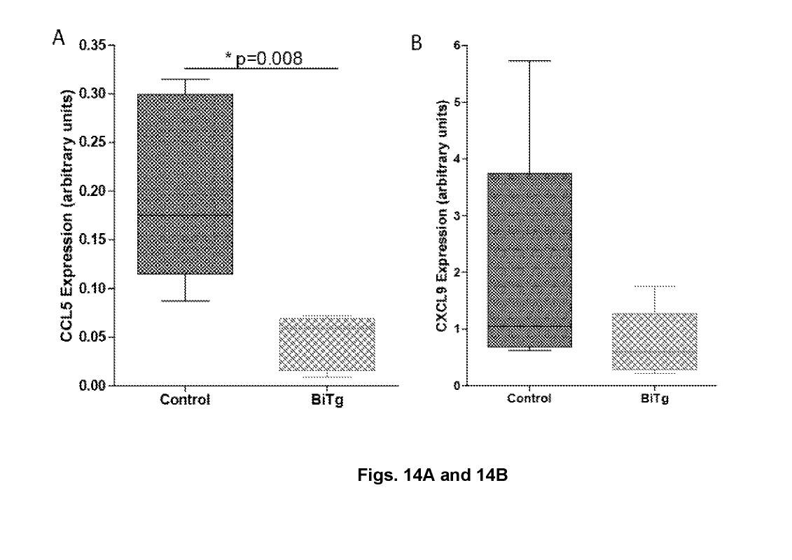

FIG. 14 illustrates that thymic epithelial Her2 activation and subsequent thymus atrophy causes reduced CCL5 and CXCR9 gene expression in non-hematopoietic thymus cells. Non-hematopoietic/non-endothelial thymus stromal cells were enriched using magnetic antibody bead separation. The present inventors found that both CCL5 and CXCL9 were reduced in gene expression in response to Her2 activation. These data agree with previous cytokine profile data (FIG. 6) and suggest that thymus epithelial Her2 activation transcriptionally represses CCL5 and CXCR9 expression. The present inventors used a minimum n=3 animals for all treatment conditions. Asterisk (*, A) denotes significance of p=0.008.

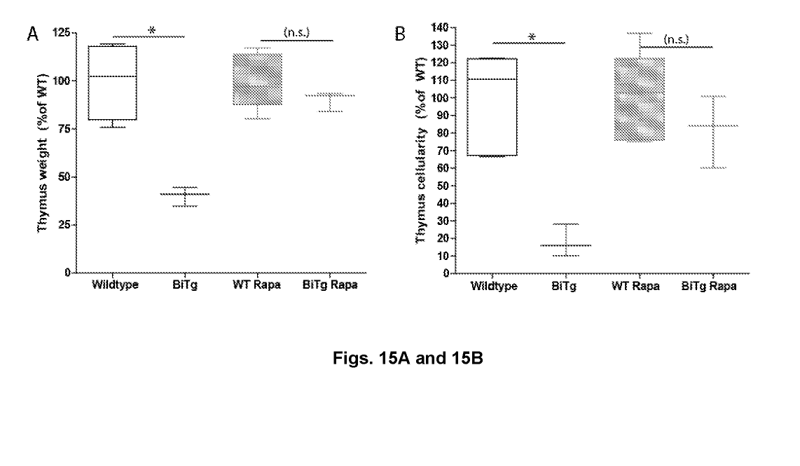

FIG. 15 illustrates that treatment with rapamycin prevents experimentally induced, epithelial Her2-dependent thymus atrophy. Adult wildtype and bitransgenic mice were treated daily with intraperitoneal rapamycin for two days prior to Her2 activation with doxycycline and throughput the three day activation time. The present inventors found that treatment with rapamycin prevented both a loss in overall thymus weight in bitransgenic mice relative to wildtype mice treated with the same compounds (A) and that rapamycin treatment prevented a loss in overall thymus cellularity in bitransgenic mice (B) when compared with doxycycline-only treated mice. The present inventors used a minimum n=3 animals for all treatment conditions. Asterisks (*, A and B) denote significance of p<0.05.

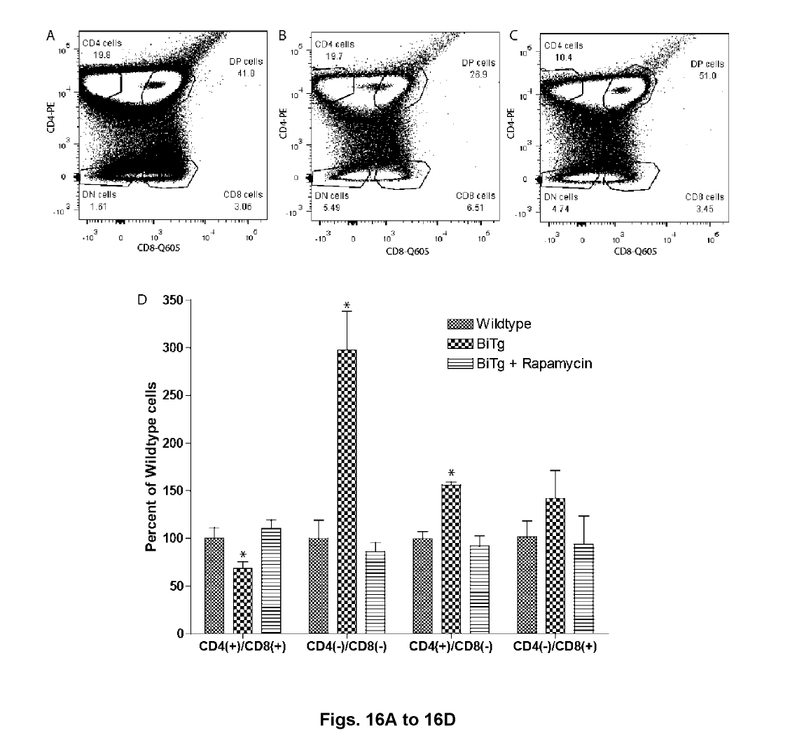

FIG. 16 illustrates that rapamycin treatment prevents Her2 activation-dependent changes to immature thymocyte abundance. The present inventors assessed immature T lymphocyte development in wildtype and BiTg mice treated with doxycycline to induce epithelial Her2 activity and in mice treated with doxycycline plus rapamycin. They examined CD4 plus CD8 expression in lineage negative thymocytes by flow cytometry (A-C) and determined that CD4 plus CD8 double positive (DP) thymocyte abundance was reduced in BiTg following doxycycline exposure but not following doxycycline plus rapamycin exposure. (D) Quantification of DP, CD4/CD8 double negative (DN), and CD4 or CD8 single positive thymocyte abundance. The present inventors used a minimum n=3 animals for all treatment conditions. Asterisks (*, D) denote significance of p,0.05.

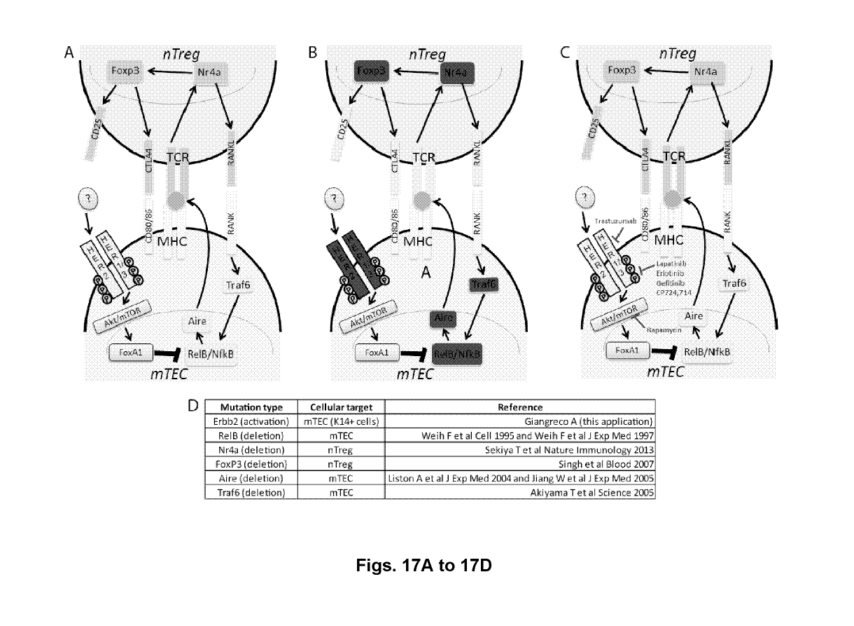

FIG. 17 illustrates a mechanistic model depicting how Her1/2 activation inhibits normal Treg, and subsequently CD4 and CD8 T cell development within the thymus. (A) represents an illustration of the overall pathway, wherein Her2 activation drives Akt/mTOR dependent FoxA1 activity that in turn represses ReIB. When Her2 activity is low, RANK/RANKL interactions drive ReIB/NfkappaB activity, Aire expression, and appropriate MHC antigen presentation (for Treg maturation and negative selection). Antigen presentation to Tregs drives Nr4a-dependent RANKL and FoxP3 expression which in turn upregulates both CD25 and CTLA4. (B) Dark grey highlighted proteins denote sites of known mutations that phenocopy the effects of Her2 activation described in this application. References to each mutation model are described in (D). (C) Her2 pathway antagonists suitable for regulating Her2 activity in the context of diseases indicated in this filing.

For FIGS. 12,13 and 16 the methods as detailed above for ‘flow cytometry and immunohistochemistry’ were used.

Materials and Method Relating to Protein and Gene Expression Assays' (for FIG. 14)

The present inventors assessed the relative expression levels of CCL5 and CXCL9 in thymus stroma cell preparations isolated using magnetic cell separation by quantitative Taqman PCR. Briefly, RNA samples were prepared using commercially available SV RNA isolation (#Z3101, Promega, USA) and qScript cDNA synthesis kits (#95048, Quanta Biosciences, UK). 500 ng of total RNA was reverse transcribed and subjected to TaqMan PCR using an Eppendorf real time PCR machine, commercially available Taqman inventoried and recommended probes (Mm01302427, Mm00434946) and and 2× PCR master mix (#4369016, Applied Biosystems). All samples were run as triplicates with a minimum of two samples per treatment type. Relative gene expression abundance was based on delta-Ct calculations and normalized using beta 2 microglobulin (Mm00437762) as a loading control transcript.

Materials And Method Relating To ‘In Vivo Experimental Procedures’ (for FIGS. 15, 16)

In some experiments, Rapamycin (20 micrograms in 200 μl PBS, #R0395, Sigma, UK) was administered by intraperitoneal injection daily for two days prior to Doxycycline administration and daily throughout the Doxycycline exposure period.

1. A therapeutic agent for use in a method for modulating the function and/or growth of a thymus in a subject, wherein the therapeutic agent comprises an HER2 or HER1 pathway antagonist or agonist, and/or a CCR/CCL5 antagonist, the method involving administering the therapeutic agent to the subject.

2. A therapeutic agent for use according to claim 1, wherein the method is for treating thymic atrophy and/or involution in the subject, and wherein the agent comprises an HER2 or HER1 pathway antagonist.

3. A therapeutic agent for use according to claim 2, wherein the agent is or comprises a compound according to formula (I) and/or an antibody that is an HER1 or HER2 pathway antagonist: 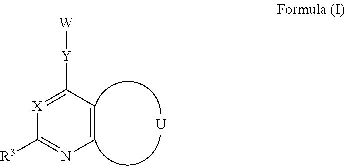
X is N, CH or C—C≡N;
Y is a group selected from NR^{a} wherein R^{a} is hydrogen or a C₁₋₈ alkyl group; CH₂, Z(CH₂), (CH₂)Z, and Z, in which Z is O, S(O)ₘ wherein m is 0, 1 or 2;
W is an optionally substituted aromatic monocyclic or aromatic bicyclic ring; 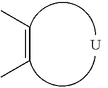
is an optionally substituted fused 5, 6 or 7-membered aromatic ring, optionally containing 1 to 5 heteroatoms which may be the same or different and which are selected from N, O or S(O)_{m′} wherein m′ is 0, 1 or 2, the heterocyclic ring containing a total of 1, 2 or 3 double bonds inclusive of the bond in the pyridine or pyrimidine ring;
R³ is selected from hydrogen, halo, trifluoromethyl, C₁₋₄ alkyl and C₁₋₄ alkoxy;
and any salt, base or prodrug form thereof.

4. An therapeutic agent for use as claimed in claim 3, wherein W is selected from any of the following optionally substituted groups: phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3-dihydro-1H-benzimidazolyl or 1H-benzotriazolyl group.

5. An therapeutic agent for use as claimed in claim 3 or 4, wherein the compound is of formula (II); 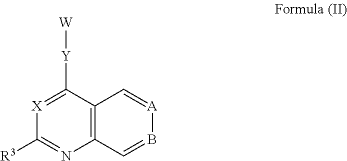
W, X, Y, Z and R³ are as defined in claim 3 or 4;
A and B are each independently selected from C—R₁, C—R₂ and CH, and at least one of A and B is C—R₁ or C—R₂;
R¹ and R² are the same or different and independently selected from halo, hydroxyl, optionally substituted C₁₋₈ alkyl, optionally substituted C₂₋₈ alkenyl, optionally substituted C₂₋₈ alkynyl, optionally substituted C₁₋₈ alkoxy, di-C₁₋₈ alkoxy, carboxy, carbonyl, C₁₋₈ alkylcarbonyl, C₁₋₈ alkoxycarbonyl, carbamoyl, alkylcarbamoyl, dialkylcarbamoyl, carbamyl, trifluoromethyl, ether, nitro, cyano, amino, hydroxyamino, aminocarbonyl, alkylamino, dialkylamino, di-[(C₁₋₄)alkyl]amino-(C₂₋₄)alkoxy, alkylaminocarbonyl, optionally substituted furyl, e.g. [(C₁₋₄)alkylsulfonyl(C₁₋₄)alkylamino)alkyl-furyl], optionally substituted phenyl, optionally substituted phenoxy, phenyl-V-alkyl, wherein V is selected from a single bond, O, S and NH, optionally substituted phenyl-(C₁₋₄)alkoxy, optionally substituted guanidine, optionally substituted ureido, optionally substituted pyridinyl, optionally substituted pyrimidinyl, optionally substituted pyrrolidinyl, pyrrolidin-1-yl-(C₂₋₄)alkoxy, optionally substituted piperidino, piperidino-(C₂₋₄)alkoxy, optionally substituted morpholino, morpholino-(C₁₋₄)alkoxy, optionally substituted piperazinyl, piperazin-1-yl(C₂₋₄)alkoxy, 4-(C₁₋₄)alkylpiperazin-1-yl-(C₂₋₄)alkoxy, optionally substituted imidazolyl, imidazol-1-yl(C₂₋₄)alkoxy, di-[(C₁₋₄)alkoxy-(C₂₋₄)alkyl]amino-(C₂₋₄)alkoxy, thiamorpholino-(C₂₋₄)alkoxy, 1-oxothiamorpholino-(C₂₋₄)alkoxy or 1,1-dioxothiamorpholino-(C₂₋₄)alkoxy, alkylthio, alkylsulphinyl, alkylsulphonyl, (E)-dimethylamino(but-2-enamide), optionally substituted (tetrahydro-furan-3-yl)-oxy;
and any salt, base or prodrug form thereof.

6. A therapeutic agent for use as claimed in any one of claims 3 to 5, wherein the compound is of formula (III); 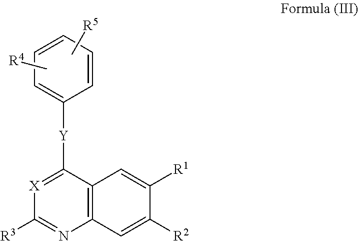
X, Y, Z and R³ are as defined in claim 2;
R¹, R² are as defined in claim 5;
R⁴ and R⁵ are the same or different and independently selected from hydrogen, halo, hydroxyl, optionally substituted C₁₋₈ alkyl, optionally substituted C₂₋₈ alkenyl, optionally substituted C₂₋₈ alkynyl, optionally substituted C₁₋₈ alkoxy, di-C₁₋₈ alkoxy, carboxy, carbonyl, C₁₋₈ alkylcarbonyl, C₁₋₈ alkoxycarbonyl, carbamoyl, alkylcarbamoyl, dialkylcarbamoyl, carbamyl, trifluoromethyl, ether, nitro, cyano, amino, hydroxyamino, aminocarbonyl, alkylamino, dialkylamino, di-[(C₁₋4)alkyl]amino-(C₂₋₄)alkoxy, alkylaminocarbonyl, optionally substituted furyl e.g. [(C₁₋₄)alkylsulfonyl(C₁₋₄)alkylamino)alkyl-furyl], optionally substituted phenyl, optionally substituted phenyl (C₁₋₈)alkoxy, optionally substituted phenoxy, phenyl-V-alkyl, wherein V is selected from a single bond, O, S and NH, optionally substituted phenyl-(C₁₋₄)alkoxy, optionally substituted guanidine, optionally substituted ureido, optionally substituted pyridinyl, optionally substituted pyrimidinyl, optionally substituted pyrrolidinyl, pyrrolidin-1-yl-(C₂₋₄)alkoxy, optionally substituted piperidino, piperidino-(C₂₋₄)alkoxy, optionally substituted morpholino, morpholino-(C₁₋₄)alkoxy, optionally substituted piperazinyl, piperazin-1-yl(C₂₋₄)alkoxy, 4-(C₁₋₄)alkylpiperazin-1-yl-(C₂₋₄)alkoxy, optionally substituted imidazolyl, imidazol-1-yl(C₂₋₄)alkoxy, di-[(C₁₋₄)alkoxy-(C₂₋₄)alkyl]amino-(C₂₋₄)alkoxy, thiamorpholino-(C₂₋₄)alkoxy, 1-oxothiamorpholino-(C₂₋₄)alkoxy or 1,1-dioxothiamorpholino-(C₂₋₄)alkoxy, alkylthio, alkylsulphinyl, alkylsulphonyl.

7. A therapeutic agent for use as claimed in any one of claims 3 to 6, wherein X is N or C—C≡N.

8. A therapeutic agent for use as claimed in any one of claims 3 to 7, wherein Y is NH.

9. A therapeutic agent for use as claimed in any one of claims 3 to 8, wherein R³ is hydrogen.

10. A therapeutic agent for use as claimed in any one of claims 5 to 9, wherein R¹ is 5-[(2-methylsulfonylethylamino)methyl]-2-furyl.

11. A therapeutic agent for use as claimed in any one of claims 5 to 10, wherein R¹ is methoxy and R² is (3-morpholin-4ylpropoxy).

12. A therapeutic agent for use as claimed in any one of claims 5 to 11, wherein R¹ and R² are 2-methoxyethoxy.

13. A therapeutic agent for use as claimed in any one of claims 1 to 9, wherein the agent comprises N-[3-chloro-4-[(3-flurophenyl)methoxy]phenyl]-6-[5-[(2-methylsulfonylethylamino)methyl]-2-furyl]quinazolin-4-amine.

14. A therapeutic agent for use as claimed in any one of claims 1 to 9, wherein the agent comprises N-(3-chloro-4-fluoro-phenyl)-7-methoxy-6-(3-morpholin-4-ylpropoxy)quinazolin-4-amine.

15. A therapeutic agent for use as claimed in any one of claims 1 to 9, wherein the agent comprises N-(3-ethynylphenyl)-6,7-bis(2-methoxyethoxy)quinazolin-4-amine.

16. A therapeutic agent for use as claimed in any one of claims 1 to 9, wherein the agent comprises (2E)-N-[4-[[3-chloro-4-[(pyridin-2-yl)methoxy]phenyl]amino]-3-cyano-7-ethoxyquinolin-6-yl]-4-(dimethylamino)but-2-enamide.

17. A therapeutic agent for use as claimed in any one of claims 1 to 9, wherein the therapeutic agent comprises N-[4-[(3-Chloro-4-fluorophenyl)amino]-7-[[(3S)-tetrahydro-3-furanyl]oxy]-6-quinazolinyl]-4(dimethylamino)-2-butenamide.

18. A therapeutic agent for use as claimed in claims 1 or claim 2, wherein the agent is selected from rapamycin and perifosine, and any salt, base or prodrug form thereof.

19. A therapeutic agent for use according to claim 3, wherein the antibody is selected from cetuximab, trastuzumab, peruzumab and panitumumab.

20. A therapeutic agent for use according to any one of the preceding claims, wherein the subject is suffering from thymic atrophy and/or involution and another disorder.

21. A therapeutic agent for use according to claim 20, wherein the other disorder is selected from a viral infection and a bacterial infection.

22. A therapeutic agent for use according to claim 21, the other disorder is a bacterial infection selected from bacterial pneumonia, methicillin-resistant Staphylococcus aureus (MRSA), Clostridium difficile and vancomycin-resistant enterococcus (VRE).

23. A therapeutic agent for use according to claim 21, the other disorder is a viral infection selected from influenza, respiratory syncytial virus and a herpes virus such as herpes zoster.

24. A therapeutic agent for use according to claim 20, the other disorder is selected from HIV, AIDS, X-linked autoimmunity and allergic dysregulation (XLAAD); Autoimmune polyendocrine syndrome type 1 (APECED), DiGeorge syndrome and Systemic Lupus Erythematosus.

25. A therapeutic agent for use according to claim 20, wherein the subject is receiving or has received a vaccine for the other disorder.

26. A therapeutic agent for use according to any one of the preceding claims, wherein the subject is immunocompromised.

27. A therapeutic agent for use according to any one of the preceding claims, wherein the subject is a human of 50 years of age or more.

28. A therapeutic agent for use according to any one of the preceding claims, wherein the subject is a human of 60 years of age or more.

29. A therapeutic agent for use according to any one of the preceding claims, wherein the subject is a human of 70 years of age or more.

30. A therapeutic agent according to claim 1, wherein the agent is a HER2 or HER1 pathway agonist and/or a CCR/CCL5 antagonist for treating a hyperactive thymus and/or excessive thymic growth in a subject.

31. A therapeutic agent according to claim 30, wherein the agent is maraviroc.

32. A therapeutic agent according to claim 30 or 31, wherein the subject is also suffering from a disorder selected from thymoma, myasthenia Gravis, thymic carcinoma, X-linked autoimmunity and allergic dysregulation (XLAAD); Autoimmune polyendocrine syndrome type 1 (APECED), DiGeorge syndrome and Systemic Lupus Erythematosus.

33. A pharmaceutical composition comprising the therapeutic agent according to any one of the preceding claims and a pharmaceutically acceptable carrier or excipient, wherein the composition is for use in a method for modulating the function and/or growth of a thymus in a subject, the method involving administering the therapeutic agent to the subject.

34. The pharmaceutical composition according to claim 33, wherein the composition is for treating thymic atrophy and/or involution in a subject, and the therapeutic agent is or comprises an HER2 or HER1 pathway antagonist.

35. The pharmaceutical composition according to claim 34, wherein the composition is for treating a hyperactive thymus and/or excessive thymic growth in a subject, and the therapeutic agent is or comprises a HER2 or HER1 pathway antagonist or agonist and/or a CCR/CCL5 antagonist.

36. A method for modulating the function and/or growth of a thymus in a subject, the method involving administering a therapeutic agent to the subject, wherein the therapeutic agent comprises an HER2 or HER1 pathway antagonist or agonist and/or a CCR/CCL5 antagonist.

37. A method according to claim 36, wherein the method is for treating thymic atrophy and/or involution in the subject, and wherein the agent comprises an HER2 or HER1 pathway antagonist.

38. A method according to claim 36, wherein the method is for treating a hyperactive thymus and/or excessive thymic growth in a subject, and the therapeutic agent is or comprises a HER2 or HER1 pathway agonist and/or a CCR/CCL5 antagonist.

39. A therapeutic agent for use in a method for treating a disorder in a subject, the disorder selected from systemic autoimmunity, peripheral autoimmunity and Systemic Lupus Erythematosus, wherein the therapeutic agent comprises an HER2 or HER1 pathway antagonist or agonist the method involving administering the therapeutic agent to the subject.

40. A therapeutic agent for use, wherein the therapeutic agent is as defined in any one of claims 2 to 19.
